SEQUENCE LISTING (6 sequences)

SEQ ID NO: 1 - DNA, 19 bp, artificial sequence
  synthetic oligonucleotide
gcagccgtaacttgcaatc

SEQ ID NO: 2 - DNA, 21 bp, artificial sequence
  synthetic oligonucleotide
cacagcgtaattcttcggacc

SEQ ID NO: 3 - DNA, 19 bp, artificial sequence
  synthetic oligonucleotide
gctttcccaaattcaccgt

SEQ ID NO: 4 - DNA, 21 bp, artificial sequence
  synthetic oligonucleotide
cacagcgtaattcttcggacc

SEQ ID NO: 5 - DNA, 23 bp, artificial sequence
  synthetic oligonucleotide
aactctcccgctatgtatgtcgc

SEQ ID NO: 6 - DNA, 23 bp, artificial sequence
  synthetic oligonucleotide
ccatctcctgctcgtagtcaaca

(19) United States
(12) Patent Application Publication
(10) Pub. No.: US 2014/0123342 A1
(43) Pub. Date: May 1, 2014

(54) Regulation of Stomatal Apertures by Apyrases and Extracellular Nucleotides

(71) Applicants: Stanley J. Roux, Austin, TX (US); Iris Steinebrunner, Dresden (DE); Gregory B. Clark, Austin, TX (US)

(72) Inventors: Stanley J. Roux, Austin, TX (US); Iris Steinebrunner, Dresden (DE); Gregory B. Clark, Austin, TX (US)

(73) Assignee: BOARD OF REGENTS, THE UNIVERSITY OF TEXAS SYSTEM, Austin, TX (US)

(21) Appl. No.: 14/118,787

(22) PCT Filed: Jun. 1, 2012
(86) PCT No.: PCT/US12/40382

§ 371 Date: Jan. 16, 2014

Related U.S. Application Data
(60) Provisional application No. 61/492,748, filed on Jun. 2, 2011.

(51) Int. Cl. C12N 15/82

(52) U.S. Cl. CPC C12N 15/8279; C12N 15/8273
USPC 800/279; 536/23.1; 536/26.7; 504/196; 504/335; 534/727; 544/208; 504/127; 504/133; 530/389.8; 564/84; 800/286; 536/24.5; 800/298; 504/333; 800/301

(57) ABSTRACT

The role of extracellular nucleotides and apyrase enzymes in the guard cells that border stomata in regulating stomatal aperture and the plant's resistance to drought and pathogens is disclosed herein. Expression of apyrases APY1 and APY2, in guard cell protoplasts is strongly correlated with cell growth, cell secretory activity and with conditions that favor stomatal opening. Both short-term inhibition of ectoapyrase activity and long-term suppression of APY1 and APY2 transcript levels significantly disrupt normal stomatal behavior in light. Furthermore, two punnoceptor inhibitors in mammals, pyridoxalphosphate-6-azo-phenyl-2′,4′-disulphonic acid (PPADS) and Reactive Blue 2, block ATPS- and ADPβS-induced opening and closing, and also partially block the ability of abscisic acid (ABA) to induce stomatal closure, and light-induced stomatal opening. Treatment of epidermal peels with ATPyS induces increased levels of nitric oxide and reactive oxygen species, and genetically suppressing the synthesis of these agents blocks the effects of nucleotides on stomatal aperture.

### STATEMENT OF FEDERALLY FUNDED RESEARCH

This invention was made with U.S. Government support under Contract Nos. IOS 0718890 and IOS 1027514 awarded by the National Science Foundation (NSF). The government has certain rights in this invention.

### TECHNICAL FIELD OF THE INVENTION

The present invention relates in general to the field of plant physiology, and more particularly, to regulating plant's resistance to drought and to pathogens by regulating apyrase activity in the guard cells that border stomata.

### REFERENCE TO A SEQUENCE LISTING

The present application includes a Sequence Listing filed separately as required by 37 CFR 1.821-1.825.

### BACKGROUND OF THE INVENTION

Without limiting the scope of the invention, its background is described in connection with to physiological methods for regulating plants resistance to drought and pathogens.

U.S. Patent Publication No. 2008/0274888 (Goldstein, 2008) relates to a potent antioxidant N-acetylcysteine amide (NAC amide), or a physiologically acceptable derivative, salt, or ester thereof, that is topically or exogenously applied to a plant, or part thereof, to reduce or prevent adverse reactions of plants and crops to environmental biotic and abiotic stresses, such as extremes of temperature, drought, humidity, frost, rain, as well as the presence or invasion of a variety of pests and pathogens. Such environmental stresses can result in oxidative stress and the correlated production (and buildup) of free radicals in plant cells, which damages plant cells and tissues and can lead to plant death. NAC amide reduces, prevents, alleviates, or otherwise counteracts such oxidative stress and free radical production, which adversely effect the overall growth and viability of the plant.

U.S. Patent Publication No. 2009/0328246 (Ramirez et al. 2009) relates to the technical field of plant biotechnology and, more specifically, to the use of the ocp3 gene as a regulator of drought resistance in plants and to the resulting plants having said drought resistance or increased drought tolerance. According to the Ramirez invention plants obtained with decreased expression of the OCP3 gene, having a large increase of the resistance to prolonged drought periods.

U.S. Patent Publication No. 2008/0254986 (Silverman et al. 2008) is directed to the use of piperonyl butoxide (PBO), alone or with S-(+)-abscisic acid (ABA) or its salts to minimize stress to plants. According to the Silverman invention the combination of PBO and ABA effectively reduced stomatal conductance in a dose-dependent manner and thereby slowed and reduced incidence of wilting.

WIPO Patent Application No. WO/2010/065725 issued to Roux et al. (2010) discloses compositions and methods of modulating the length of one or more cotton fibers in a plant by contacting the plant or tissue derived therefrom with at least one of: a nucleotide; a modulator of ectoapyrase gene transcription; or an anti-ectoapyrase antibody or fragments thereof, at a concentration that modulates growth of one or more cotton fibers.

### SUMMARY OF THE INVENTION

The present invention describes the discovery that apyrase enzymes and extracellular nucleotides such as ATP, ADP, ATP-Gamma-S and ADP-Beta-S can regulate the opening and closing of stomatal pores, which plants use for carbon dioxide uptake and water transpiration. The present invention further discloses regulating apyrase activity in the guard cells that border stomata could regulate plant's resistance to drought and to pathogens.

An agricultural composition for increasing a resistance or a tolerance of a plant to one or more environmental stress conditions is described in one embodiment of the present invention. The composition comprises at least one of an extracellular exogenous nucleotides selected from di-nucleotides, tri-nucleotides, or poorly-hydrolyzable nucleotides, an ectopyrase activity inhibitor, a modulator of an ectoapyrase gene transcription, an anti-ectoapyrase antibody or fragments thereof at a concentration sufficient to increase the resistance or the tolerance of the plant to the one or more environmental stress conditions. In one aspect of the composition of the present invention the one or more poorly-hydrolyzable nucleotides comprise thio, methylene, amide or methyl-modified ATP, ADP, UTP, UDP, CTP, CDP, TTP, TDP, GTP, GDP, dATP, dADP, dUTP, dUDP, dCTP, dCDP, dTTP, dTDP, dGTP, dGDP, ATPγS, ADPβS, and analogues and combinations thereof. In a specific aspect the one or more poorly-hydrolyzable nucleotides comprise ATPγS, ADPβS, or both.

In another aspect the composition modulates or suppresses the activity, the expression or both of one or more apyrase enzymes, wherein the apyrase enzyme comprises APY1, APY2, or both. In yet another aspect the environmental stress conditions are selected from the group consisting of extreme temperature or weather conditions, drought, frost, rain, hail, moisture, humidity, heat, excess salinity, excess minerals, poor soil nutrients, poor growth medium nutrients, insects, arachnids, nematodes, and other pest infestations, bacteria, fungi, viruses, mycoplasma, and other pathogens, and other biotic or abiotic environmental stress conditions. In related aspects the environmental stress conditions is drought or a pathogenic infestation.

In another aspect the composition is administered to a surface of the plant, wherein the surface of the plant comprises foliage, leaves, stems, roots, flowers, buds, and stalks. In another aspect the composition is administered to the leaf of the plant. In a related aspect composition is administered by spraying the plant, direct application to the surface of the plant, pouring onto the plant, drenching the root system of the plant, administered as a mixture with soil, water, nutrients, or manure, or any combinations thereof. In yet another aspect the composition increases the resistance or the tolerance of a plant to one or more environmental stress conditions by regulating the opening or the closing of one or more stomatal pores and modulates extracellular ATP (eATP) mediated cell signaling in the plant.

Another embodiment of the instant invention relates to a method for increasing a resistance or a tolerance of a plant to one or more environmental stress conditions comprising the step of administering to the surface of the plant an agricultural composition comprising at least one of an extracellular exogenous nucleotides selected from di-nucleotides, tri-nucleotides, or poorly-hydrolyzable nucleotides, an ectopyrase activity inhibitor, a modulator of an ectoapyrase gene transcription, an anti-ectoapyrase antibody or fragments thereof in an amount effective to induce resistance or tolerance in the plant against the one or more environmental stress conditions. In one aspect of the method the one or more poorly-hydrolyzable nucleotides comprise thio, methylene, amide or methyl-modified ATP, ADP, UTP, UDP, CTP, CDP, TTP, TDP, GTP, GDP, dATP, dADP, dUTP, dUDP, dCTP, dCDP, dTTP, dTDP, dGTP, dGDP, ATPγS, ADPβS, and analogues and combinations thereof. In another aspect the one or more poorly-hydrolyzable nucleotides comprise ATPγS, ADPβS, or both. In yet another aspect of the method the composition modulates or suppresses the activity, the expression or both of one or more apyrase enzymes, wherein the apyrase enzyme comprises APY1, APY2, or both.

In a related aspect the environmental stress conditions are selected from the group consisting of extreme temperature or weather conditions, drought, frost, rain, hail, moisture, humidity, heat, excess salinity, excess minerals, poor soil nutrients, poor growth medium nutrients, insects, arachnids, nematodes, and other pest infestations, bacteria, fungi, viruses, mycoplasma, and other pathogens, and other biotic or abiotic environmental stress conditions. In one aspect the surface of the plant comprises foliage, leaves, stems, roots, flowers, buds, and stalks. In another aspect the composition is administered to the leaf of the plant by spraying the plant, direct application to the surface of the plant, pouring onto the plant, drenching the root system of the plant, administered as a mixture with soil, water, nutrients, or manure, or any combinations thereof. In yet another aspect the composition increases the resistance or the tolerance of a plant to one or more environmental stress conditions by regulating the opening or the closing of one or more stomatal pores.

In yet another embodiment the instant invention discloses a composition for regulating opening or closing of one or more stomatal pores in a plant comprising at least one of an extracellular exogenous nucleotides selected from di-nucleotides, tri-nucleotides, or poorly-hydrolyzable nucleotides at a concentration sufficient to regulate the opening or the closing of the one or more stomatal pores.

In one embodiment the instant invention describes a method for regulating opening or closing of one or more stomatal pores in a plant comprising the step of administering to a leaf surface of the plant an agricultural composition comprising at least one of an extracellular exogenous nucleotides selected from di-nucleotides, tri-nucleotides, or poorly-hydrolyzable nucleotides in an amount effective to regulate the opening or the closing of one or more stomatal pores in the plant. In one aspect of the method the one or more poorly-hydrolyzable nucleotides comprise ATPγS, ADPβS, or both, wherein the ATPγS, ADPβS, or both are administered to induce an opening of the one or more stomatal pores to increase a photosynthesis rate by increased CO₂ uptake from the environment. In a related aspect the ATPγS, ADPβS, or both are administered at a concentration ranging from 5 μM to 15 μM to induce opening of the one or more stomatal pores. In another aspect the ATPγS, ADPβS, or both are administered to induce a closure of the one or more stomatal pores to confer increased resistance or tolerance to brought conditions, pathogens, or both. In yet another aspect the ATPγS, ADPβS, or both are administered at a concentration ranging from 150 μM to 200 μM or higher to induce closure of the one or more stomatal pores.

In another embodiment the present invention provides a composition to modulate an extracellular exogenous nucleotide regulated opening or closing of one or more stomatal pores in a plant comprising at least one of a mammalian purinoreceptor antagonist, wherein the mammalian purinoreceptor antagonist is selected from the group consisting of pyridoxalphosphate-6-azo-phenyl-2′,4′-disulphonic acid (PPADS), Reactive Blue 2 (RB-2), or both. In one aspect the extracellular exogenous nucleotides are selected from the group consisting of di-nucleotides, tri-nucleotides, or poorly-hydrolyzable nucleotides. In another aspect the composition may comprise one or more additional mammalian purinoreceptor antagonists selected from the group consisting of 8′[carbonylbis(imino-3,1-phenylcarbonylimino)]bus-1,3,5-naphthalene-trisulphonic acid (NF023), 2′,3′-O-(2,4,6-trinitrophenyl)-ATP (NT-ATP), pyridoxal-a5-phosphate-6-phenylazo-4′-carboxylic acid (MRS2159), 8,8′-(carbonylbis(imino-4,1-phenylenecarbonyl-imino-4,1-phenylenecarbonylimino))bis(1,3,5-naphthalenetrisulfonic acid) (NF279), 4,4′,4″,4′″-[carbonylbis(imino-5,1,3-benzenetriyl-bis(carbonylimino))]tetrakis-1,3-benzenedisulfonic acid (NF449), brilliant blue G (BB-G), 4′,4″,4′″-[carbonylbis(imino-5,1,3-benzenetriyl bis(carbonylimino))]tetrakisbenzenesulfonic acid (NF110), 1-[N,O-bis(5-Isoquinolinesulfonyl)-N-methyl-L-tyrosyl]-4-phenylpiperazine (KN-62), 3-[[5-(2,3-Dichlorophenyl)-1H-tetrazol-1-yl]methyl]pyridine (A438079), 2′deoxy-N⁶-methyladenosine-3′5′-biphosphate (MRS2179), 2-chloro-N⁶-methyl-(N)-methanocarba-2′-deoxyadenosine3′,5′-biphosphate (MRS2279), 2-iodo-N6-methyl-(N)-methanocarba-2′-deoxyadenosine3′,5′-biphosphate (MRS2500), N,N″-1,4-butanediylbis[N′-(3-isothiocynatophenyl)thio urea (MRS2578), 8,8′-[carbonylbis[imino-3,1-phenylenecarbonylimino(4-fluoro-3,1-phenylene)carbonylimino]]bis-1,3,5-naphthalene trisulfonic acid (NF157), N6-(2-methylthioethyl)-2-(3,3,3-trifluoropropylthio)-β-γ-dichloromethylene-ATP (cangrelor), clopidogrel, 2-[(2-chloro-5-nitrophenyl)azo]-5-hydroxy-6-methyl-3[(phosphonooxymethyl]-4-pyridinecarboxaldehyde (MRS2211), UDP, and any combinations thereof. In yet another aspect the mammalian purinoreceptor antagonist blocks a dose-dependent stomatal opening or closure mediated by the extracellular exogenous nucleotides by an inhibition of extracellular ATP (eATP) mediated cell signaling in the plants.

In yet another embodiment the instant invention provides a method for modulating an extracellular exogenous nucleotide regulated opening or closing of one or more stomatal pores in a plant comprising the step of administering to a leaf surface of the plant at least one of a mammalian purinoreceptor antagonist, wherein the mammalian purinoreceptor antagonist comprises pyridoxalphosphate-6-azo-phenyl-2′,4′-disulphonic acid (PPADS), Reactive Blue 2 or both in an amount effective to modulate an extracellular exogenous nucleotide regulated opening or closing of the one or more stomatal pores.

The present invention further discloses a composition and a method to reduce water loss, transpiration, wilting or any combinations thereof in a plant due to excessive sunlight, artificial light, heat or any combinations thereof. The composition comprises at least one of a mammalian purinoreceptor antagonist, wherein the mammalian purinoreceptor antagonist comprises pyridoxalphosphate-6-azo-phenyl-2′,4′-disulphonic acid (PPADS), Reactive Blue 2 (RB-2) or both. The composition acts by partially blocking a light induced opening of the one or more stomatal pores when provided in an amount effective to reducing water loss, transpiration, wilting or any combinations thereof.

One embodiment of the present invention is related to an agricultural composition for increasing a resistance or a tolerance to drought, pathogens or both comprising at least one of a chemical ectopyrase activity inhibitor, a modulator of an ectoapyrase gene transcription, an anti-ectoapyrase antibody or fragments thereof at a concentration sufficient to increase the resistance or the tolerance of the plant to drought, pathogens or both. In one aspect the composition inhibits the activity, the expression or both of one or more apyrase enzymes, wherein the apyrase enzymes comprise APY1, APY2, or both. In another aspect the chemical ectopyrase activity inhibitor is selected from the group consisting of N-(3-methylphenyl)-4-biphenylsulfonamide (NXGT191), N′-(2-hydroxy-5-methylbenzylidene)-2-(1-naphthyl)acetohydrazide, 3-{[(4-bromophenyl)amino]sulfonyl}-N-(3-nitrophenyl)benzamide (NXGT1913), or any combinations thereof. In yet another aspect the chemical ectopyrase activity inhibitor comprises N-(3-methylphenyl)-4-biphenyl sulfonamide (NXGT191).

In another aspect the inhibition of the expression of the APY1, APY2, or both is done by RNA interference (RNAi) using one or more anti-sense or siRNA gene inhibitors. In yet another aspect the composition increases the resistance or the tolerance of the plant to drought, pathogens or both by blocking an apyrase enzyme induced opening of one or more stomatal pores in the plant when administered by spraying, pouring, or direct application to a leaf surface of the plant. In a specific aspect the plant is Arabidopsis.

Another embodiment of the present invention relates to a method for increasing a resistance or a tolerance to drought, pathogens or both in a plant comprising the step of administering to a leaf surface of the plant an agricultural composition comprising at least one of a chemical ectopyrase activity inhibitor, a modulator of an ectoapyrase gene transcription, an anti-ectoapyrase antibody or fragments thereof at a concentration sufficient to increase the resistance or the tolerance of the plant to drought, pathogens or both. In one aspect the composition inhibits the activity, the expression or both of one or more apyrase enzymes, wherein the apyrase enzymes comprise APY1, APY2, or both by RNA interference (RNAi) using one or more anti-sense or siRNA gene inhibitors. In another aspect the chemical ectopyrase activity inhibitor comprises NXGT191. In yet another aspect the composition increases the resistance or the tolerance of the plant to drought, pathogens or both by blocking an apyrase enzyme induced opening of one or more stomatal pores in the plant.

The present invention further discloses a method of conferring increased resistance or tolerance to drought, pathogens or both to a plant comprising the step of: (i) identifying the plant in need of increased resistance or tolerance to drought, pathogens or both and (ii) modifying the plant genetically by partially suppressing the transcription of an APY1 gene or an APY2 gene using RNAi, wherein the genetic modification results in a decreased stomatal aperture in the plant resulting in increased resistance or tolerance to drought, pathogens or both. The present invention also discloses a genetically modified plant with increased resistance or tolerance to drought, pathogens or both made by the method described hereinabove.

A transgenic plant with increased resistance or tolerance to drought, pathogens or both is disclosed in one embodiment of the present invention. The transgenic plant has a decreased transcription of an APY1 gene or an APY2 gene and a decreased stomatal aperture.

In another embodiment the instant invention provides a cocktail for promoting increased resistance or tolerance to drought pathogens or both comprising: (i) an extracellular exogenous nucleotides selected from di-nucleotides, tri-nucleotides, or poorly-hydrolyzable nucleotides, (ii) at least one of an ectopyrase activity inhibitor, a modulator of an ectoapyrase gene transcription, an anti-ectoapyrase antibody or fragments, or any combinations thereof, and (iii) a dispersion medium comprising the nucleotides, the inhibitor or both, wherein the dispersion medium comprises an aqueous solvent, an organic solvent, a gas foam, a propellant, or any combinations thereof. In one aspect of the cocktail of the present invention the one or more poorly-hydrolyzable nucleotides comprise ATPγS, ADPβS, or both. In another aspect the cocktail modulates or suppresses the activity, the expression or both of one or more apyrase enzymes, wherein the apyrase enzyme comprises APY1, APY2, or both when administered to a surface of the plant, wherein the surface of the plant comprises foliage, leaves, stems, roots, flowers, buds, and stalks. In yet another aspect the cocktail increases the resistance or the tolerance of a plant to the drought, the pathogens, or both by regulating the opening or the closing of one or more stomatal pores and modulates the extracellular ATP (eATP) mediated cell signaling in the plant.

In yet another embodiment the instant invention discloses a method of increasing resistance or tolerance to drought, pathogens or both in a plant comprising the steps of: i) identifying the plant in need of increased resistance to drought, pathogens or both; and ii) administering to the surface of the plant a cocktail comprising: a) an extracellular exogenous nucleotides selected from di-nucleotides, tri-nucleotides, or poorly-hydrolyzable nucleotides, b) at least one of an ectopyrase activity inhibitor, a modulator of an ectoapyrase gene transcription, an anti-ectoapyrase antibody or fragments, or any combinations thereof, and c) a dispersion medium comprising the nucleotides, the inhibitor or both, wherein the dispersion medium comprises an aqueous solvent, an organic solvent, a gas foam, a propellant, or any combinations thereof.

### BRIEF DESCRIPTION OF THE DRAWINGS

For a more complete understanding of the features and advantages of the present invention, reference is now made to the detailed description of the invention along with the accompanying figures and in which:

FIGS. 1A and 1B show apyrase expression is enriched in preparations of guard cell protoplasts compared to extracts of whole leaves: (FIG. 1A) Assayed by RT-PCR, APY1 and APY2 transcripts are present at a higher level in guard cell protoplast preparations compared to extracts of whole leaves. Control levels of an actin PCR product indicate equal amounts of cDNA as starting material prior to PCR and (FIG. 1B) Immunoblot analysis using anti-APY1 antibodies shows that immunodetectable protein levels of APY1/2 are higher in guard cell protoplast preparations compared to extracts of whole leaves. Control levels of α-tubulin show equal loading of protein. Leaves taken from three-week old plants grown under identical conditions were used for both the protoplast preparations and the whole leaf extracts;

FIGS. 2A and 2B show that open stomata have more active APY1/2 promoters and light-treated guard cell protoplasts have higher APY1/2 protein levels: (FIG. 2A) APY1:GUS and APY2:GUS plants were grown in low humidity (33% relative humidity) and high (85% relative humidity) conditions. Leaves were harvested after 7 hours of light (“day”) and after 4 hours in the dark (“night”) and stained for GUS activity. Bright-field images of the abaxial epidermis of whole mount leaves from the APY2:GUS line 3-2-11 are shown representing the staining pattern of all four GUS lines analysed. A dashed line marks the outlines of some weakly stained guard cells in the top right panel. The scale bars represent 100 am and (FIG. 2B) Western blot analysis of APY1/APY2 protein levels in dark-adapted guard cell protoplasts after treatment with light at various time points. Treatment with light for 15 min results in an increase in immunodetectable APY1/APY2 protein levels;

FIGS. 3A and 3B show chemical and immunological inhibition of apyrase activity induces stomatal closure: (FIG. 3A) Application of anti-apyrase immune sera induced stomatal closure in whole leaves but application of control pre-immune sera had no effect on the aperture and (FIG. 3B) Application of apyrase inhibitor NGXT191 induced stomatal closure in epidermal peels and 100 μM PPADS blocked this closure, but 100 μM PPADS had no effect alone. Apertures measured as width/length after 1 h treatment for peels and 2 h treatment for leaves. Error bars represent standard error. Different letters above the bars indicate mean values that are significantly different from one another (p<0.05; n≧50). The data presented herein are representative of three or more biological repeats;

FIGS. 4A and 4B show the dose-response curves for the effects of various concentrations of ATPγS on stomatal aperture in epidermal peel studies: (FIG. 4A) Treatment with 10 μM ABA induced stomatal closure in the light, as did 200 μM and 250 μM ATPγS. Treatment with 150 μM ATPγS or 250 μM AMPS had no statistically significant effect on stomatal aperture and (FIG. 4B) Treatment with 1 h of light induced stomatal opening, and application of 5 and 15 μM ATPγS induced stomatal opening in darkness. Treatment with 15 μM AMPS had no effect on stomatal aperture. Apertures measured as width/length after 1 h treatment. Error bars represent standard error. Different letters above the bars indicate mean values that are significantly different from one another (p<0.05; n≧50). The data presented herein are representative of three or more biological repeats;

FIGS. 5A and 5B show the animal purinergic receptor antagonist, PPADS, blocks ATPγS-induced changes in stomatal aperture and partially blocks the effects of ABA and light on stomatal aperture: (FIG. 5A) Treatment with 200 μM ATPγS induced stomatal closure in leaves and co-treatment with 100 μM PPADS blocked this closure but 100 μM PPADS alone had no effect on stomatal aperture. Treatment with 10 μM ABA induces stomatal closure and co-treatment with 100 μM PPADS partially blocked this ABA-induced stomatal closing and (FIG. 5B) Treatment with 15 μM ATPγS induced stomatal opening in epidermal peels and co-treatment with 100 μM PPADS blocked this opening but 100 μM PPADS alone had no effect on stomatal aperture. Treatment with light induced stomatal opening, however co-treatment with 100 μM PPADS partially blocked this light-induced stomatal opening. Apertures measured as width/length after 1 h treatment for peels and 2 h treatment for leaves. Error bars represent standard error. Different letters above the bars indicate mean values that are significantly different from one another (p<0.05; n≧50). The data presented herein are representative of three or more biological repeats;

FIGS. 6A and 6B show that RNAi suppression of APY1 in an APY2 single knockout results in increased stomatal apertures compared to WS wild-type. Treatment with light and 10 μM ABA induces more open stomata in leaves of RNAi plants treated with estradiol compared to leaves of WS wild-type plants treated with estradiol. Apertures measured as width/length after 2 h treatment. Error bars represent standard error. Different letters above the bars indicate mean values that are significantly different from one another (p<0.05; n≧50). The data presented herein are representative of three or more biological repeats;

FIGS. 7A-7D show that high concentrations of ATPγS induce stomatal closure via increased levels of NO and H₂O₂ in guard cells: (FIG. 7A) Treatment of wild-type leaf tissue with 10 μM ABA or 200 μM ATPγS induces a differential accumulation of H₂DCFDA fluorescence at 30 min in guard cells compared to control tissue. Different letters above the bars indicate mean values that are significantly different from one another (p<0.05; n≧25). The data in FIG. 7A is representative of three biological repeats, (FIG. 7B) Treatment of wild-type leaf tissue with 10 μM ABA or 200 μM ATPγS induces a differential accumulation of DAF-2 DA fluorescence at 45 min in guard cells compared to control tissue, (FIG. 7C) Treatment with 200 μM ATPγS and 10 μM ABA induced stomatal closure in wild-type leaves but not in atrbohD/F leaves, and (FIG. 7D) Treatment with 200 μM ATPγS and 10 μM ABA induced stomatal closure in wild-type leaves but not in nia1nia2 leaves. Different letters above the bars indicate mean values that are significantly different from one another (p<0.05; n≧25 for fluorescence studies and n≧50 for stomatal aperture studies). The data above are representative of two biological repeats;

FIGS. 8A-8D show that ABA treatment of light-adapted leaves induces release of ATP in guard cells, as assayed by ecto-luciferase luminescence: (FIG. 8A) Background levels of ecto-luciferase luminescence are observed in an epidermal peel from an untreated x-luc9 leaf (light control), (FIG. 8B) An epidermal peel from an x-luc9 leaf treated with 10 μM ABA in light for 5 min shows ecto-luciferase luminescence in guard cells, (FIG. 8C) An epidermal peel from an x-luc9 leaf treated with 1 mM ATP in light for 5 min shows ecto-luciferase luminescence in guard cells. Scale bar for A, B=50 μm and for C=100 μm. Luminescence levels are represented in pseudocolor, and (FIG. 8D) Quantification of luciferase activity from a representative data set of a closing study. Treatments with 10 μM ABA and 1 mM ATP were done in the light. Different letters above the bars indicate mean values that are significantly different from one another (p<0.05; n≧15 guard cell pairs). The data shown in FIGS. 8A-8D are representative of three biological repeats;

FIGS. 9A-9D show that the light treatment of dark-adapted leaves induces release of ATP in guard cells, as assayed by ecto-luciferase luminescence: (FIG. 9A) Background levels of ecto-luciferase luminescence are observed in an epidermal peel from an untreated x-luc9 leaf (dark control), (FIG. 9B) An epidermal peel from an x-luc9 leaf treated with 10 min of light shows ecto-luciferase luminescence in guard cells, (FIG. 9C) An epidermal peel from an x-luc9 leaf treated with 1 mM ATP in the dark shows ecto-luciferase luminescence in guard cells. Scale bar for A, B=50 μm and for C=100 μm. Luminescence levels are represented in pseudocolor, and (FIG. 9D) Quantification of luciferase activity from a representative data set of an opening study. Treatment with 1 mM ATP was done in the dark. Different letters above the bars indicate mean values that are significantly different from one another (p<0.05; n≧15 guard cell pairs). The data shown in FIGS. 9A-9D are representative of three biological repeats; and

FIG. 10 is a schematic showing a model for the regulation of stomatal movements by extracellular nucleotides. Treatment with the nucleotides ATPγS and ADPβS at high concentrations (>150-250 μM) induces stomatal closure and the release of NO and H₂O₂, whereas addition of low concentrations of these nucleotides (15-35 μM) leads to opening of stomata. These responses to either high (indicated by larger type) or low concentrations of nucleotides can be blocked by the mammalian purinoceptor inhibitor PPADS, which can also block the ability of ABA to induce stomatal closing and the ability of light to induce opening. The light treatment that induces stomatal opening also induces a higher expression of the transcripts and proteins of APY1 and APY2, and the text discusses the likelihood that these are ectoapyrases that would help regulate the concentrations of extracellular nucleotides during stomatal opening and closing.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

The term “environmental stress” as used in the present invention refers to any adverse effect on metabolism, growth or viability of the cell, tissue, seed, organ or whole plant which is produced by an non-living or non-biological environmental stressor. More particularly, it also encompasses environmental factors such as water stress (flooding, water logging, drought, dehydration), anaerobic (low level of oxygen, CO₂ etc.), aerobic stress, osmotic stress, salt stress, temperature stress (hot/heat, cold, freezing, frost) or nutrients deprivation, pollutants stress (heavy metals, toxic chemicals), ozone, high light, pathogen (including viruses, bacteria, fungi, insects and nematodes) and combinations of these.

The term “drought condition” as used herein indicates a condition under which plants can grow, but growth is suppressed because of limited humidity and water supply.

As used herein, the term “plant pathogen” refers to any organism that can cause harm to a plant. A plant can be harmed by an inhibition or slowing of the growth of a plant, by damage to the tissues of a plant, by a weakening of the immune system of a plant, by a reduction in the resistance of a plant to abiotic stresses, by a premature death of the plant, and the like.

The term “stomata” as used herein refers to pores present on the underside of the leaf. Water loss in plants, in the form of transpiration, occurs through the stomates and is controlled by the size of the stomatal opening. The greater the size of the stomatal opening, the greater is the stomatal conductance, and so transpiration (water loss) is greater.

The term “nucleotide” as used herein includes DNA and RNA, wherein they conventionally have adenine, cytosine, guanine, thymine and uracil as bases and deoxyribose and ribose as the structural sugar elements. Furthermore, a nucleotide can, however, also comprise any modified base known to current technology, which is capable of base pairing using at least one of the aforesaid bases.

The term “apyrase” as used herein refers to a nucleotide hydrolase that catalyzes the hydrolysis of nucleoside triphosphate and nucleoside diphosphate into constituent nucleoside monophosphate and phosphate. An “apyrase” has an activity described as EC 3.6.1.5, according to IUBMB enzyme nomenclature. The systematic name for an “apyrase” is ATP diphosphohydrolase (phosphate-forming).

As used herein, the term “RNA interference” (RNAi) refers to gene silencing mechanisms that involve small RNAs (including miRNA and siRNA) are frequently referred to under the broad term RNAi. Natural functions of RNAi include protection of the genome against invasion by mobile genetic elements such as transposons and viruses, and regulation of gene expression. “RNA interference” results in the inactivation or suppression of expression of a gene within an organism.

The term “gene transcription” as it is used herein means a process whereby one strand of a DNA molecule is used as a template for synthesis of a complementary RNA by RNA polymerase.

As used herein, the term “photosynthesis” is defined as the light-induced cleavage of water into molecular hydrogen and oxygen wherein the photocatalysts that participate in the reaction may be of biological or non-biological origin.

The term “transgenic plant” as used in the present invention relates to plants which have been generated using recombinant genetics and/or microbiological methods, and not by conventional breeding methods, and which contain at least one promoter. Methods for generating transgenic plants are described (Tingay S-, McElroy D., Kalla R., Fieg S., Wang M., Thorton S. and Brettel R. (1997): Agrobacterium tumefaciens-mediated barley transformation. Plant Journal 11; 1369-1376; Wan Y. and Lemaux P. (1994): Generation of a large number of independently transformed fertile barley plants. Plant Physiol. 104; 37-48, Stahl R., H. Horvath, J. Van Fleet, M. Voetz, D. von Wettstein & N. Wolf (2002) T-DNA integration into the barley genome from single and double cassette vectors. Proc. Natl. Acad., Sci. USA 99, 2146-2151; Horvath H., J. Huang, 0. T. Wong & D. von Wettstein (2002) Experiences with genetic transformation of barley and characteristics of transgenic plants. In: Barley Science, G. A. Slafer, J. L. Molina-Cano, R, Savin, J. L. Araus & I. Romagosa eds. The Harworth Press, New York 2002 pp. 143-176; Horvath H., L. G. Jensen, 0. T. Wong, E. Kohl, S, E, Ullrich, J. Cochran, C. G. Kannangara & D. von Wettstein (2001) Stability of transgene expression, field performance and recombination breeding of transformed barley lines. Theor. Appl. Genet. 102, 1-11; Wettstein D. von, G. Mikhaylenko, J. A. Froseth & C. G. Kannangara (2000) Improved barley broiler feed with transgenic malt containing heat-stable (1,3-1,4)-glucanase. Proc. Natl. Acad. Sci. USA 97, 13512-13517; Horvath H, J. Huang, 0. T. Wong, E. Kohl, T. Okita, C. G. Kannangara & D. von Wettstein (2000) The production of recombinant proteins in transgenic barley grains. Proc. Natl. Acad. Sci. USA 97, 1914-1919; Mayerhofer, R., Koncz-Kalman, Z., Nawrath, C., Bakkeren, G., Crameri, A., Angelis, K., Redei, G. P., Schell, J., Hohn, B. & Koncz, C. (1991) EMBO J. 10, 697-704 T-DNA integration: a mode of illegitimate recombination in plants; Deblaere R., Bytebier B., De Greve H., Deboeck F., Schell M., Van Montagu M., Leemans J.; “Efficient octopine Ti plasmid-derived vectors for Agrobacterium-mediated gene transfer to plants”; Nucleic Acids Res. 13:4777-4788 (1985)).

The term “enzyme inhibitor” refers to any compound that prevents an enzyme (apyrase in the present invention) from effectively carrying out its biochemical role(s). The term “antagonist” is used in its normal sense in the art, i.e., a chemical compound which prevents functional activation of a receptor by its natural agonist.

The present invention describes the role of apyrase enzymes and extracellular nucleotides such as ATP, ADP, ATP-Gamma-S and ADP-Beta-S in the regulation of the opening and closing of stomatal pores, which plants use for carbon dioxide uptake and water transpiration. The present invention further discloses methods of manipulating or regulating apyrase activity in the guard cells that border stomata and thereby modifying the plant's resistance to drought and to pathogens. The present invention for the first time shows a correlation between apyrases and/or extracellular nucleotides and stomatal pore size. The methods described herein provide agricultural and plant biotechnologists with another tool to regulate plant's resistance to drought and pathogens.

The present inventors present a non-limiting example of a plant belonging to the Arabidopsis genus to test the correlation between apyrases/extracellular nucleotides and stomatal pore size. In Arabidopsis the expression of two very similar apyrases (NTPDases), APY1 and APY2, is strongly correlated with growth and secretory activity in diverse cell types. As judged by RT-PCR, immunoblots, and promoter activity assays with glucuronidase (GUS) reporter lines, both apyrases are expressed strongly in guard cells. Immunoblots and promoter:GUS staining indicate that the guard cells of open stomates express more APY1 and APY2 than those of closed stomates. Short-term inhibition of ectoapyrase activity by polyclonal antibodies mimics ABA in inducing stomatal closure in light, whereas mutants with long-term suppression of APY1 and APY2 transcripts exhibit wider stomatal apertures in light. Dose-response tests with applied nucleotides indicate that lower concentrations (5-15 μM) of the poorly hydrolysable nucleotides ATPγS and ADPβS induce stomatal opening, but higher concentrations (150-200 μM) induce closure, while neither 15 μM nor 200 μM AMPS has any effect on stomatal aperture. All of these dosage effects are blocked by two different inhibitors of mammalian purinoceptors, pyridoxalphosphate-6-azo-phenyl-2′,4′-disulphonic acid (PPADS), and Reactive Blue 2. These antagonists also partially block the ability of abscisic acid (ABA) to induce stomatal closure, and of light to induce stomatal opening. Treatment of epidermal peels with 200 μM ATPγS induces increased levels of nitric oxide and reactive oxygen species, suggesting that these signaling agents could help mediate the effects of applied nucleotides on stomatal closure. A luciferase assay indicates that treatments that induce either the closing or opening of stomates also induce the release of ATP from guard cells. Overall these results suggest that the swelling and shrinking of guard cells induced by various stimuli may result in the release of nucleotides that help regulate stomatal apertures.

The swelling or shrinking of guard cells in the leaf epidermis controls stomatal aperture. Guard cells respond to a variety of stimuli including abscisic acid (ABA) and blue light to regulate stomatal apertures through changes in ion transport, water potential, and osmotic pressure. Swelling and shrinking events may be accompanied by changes in surface area of the plasma membrane requiring membrane exocytosis and endocytosis to accommodate the fluctuating volume (Shope et al., 2003). In recent years, guard cell signaling pathways have been elucidated, revealing new roles for nitric oxide (NO) and reactive oxygen species (ROS) in stomatal closure (Mata and Lamattina, 2001; Bright et al., 2006; Desikan et al., 2006). These findings illustrate the complexity of guard cell responses, and an understanding of the signaling pathways remains incomplete (Neill et al. 2008).

Intracellular ATP has long been known as a cellular energy source, but now extracellular ATP (eATP) has become recognized as a signaling agent in both plants and animals (Roux and Steinebrunner, 2007; Clark and Roux, 2009; Tanaka et al., 2010a). In mammals, it binds to purinergic receptors of the P2 receptor family, which induces a rapid increase in [Ca²⁺]_{cyt} that leads to diverse physiological responses (Burnstock, 2008). In plants, application of ATP also controls [Ca²⁺]_{cyt} fluctuations (Demidchik et al., 2003, 2009; Jeter et al., 2004; Tanaka et al., 2010b), although the plant receptor that initiates these responses remains unknown. As it does in animals, eATP in plants also upregulates transcripts for proteins involved in signal transduction (Jeter et al., 2004; Song et al., 2006). Downstream of the changes in [Ca²⁺]_{cyt}, but upstream of the gene expression changes, applied ATP can promote growth altering accumulation of ROS and NO in diverse tissues of diverse plants (reviewed in Tanaka et al., 2010a). These accumulations appear to be critical intermediates for eATP signaling, for genetic suppression of ROS or NO production can block cell and tissue responses to applied nucleotides (Song et al., 2006; Reichler et al., 2009; Clark et al., 2010b).

The involvement of NO and ROS in guard cell responses, and their production via eATP signaling in other plant cells, leads the present inventors to hypothesize that eATP may also play an important role in stomatal signaling pathways. An additional rationale for testing the role of eATP in stomata function was the evidence of Wolf et al. (2007) that the apyrase (NTPDase) enzymes APY1 and APY2 were strongly expressed in guard cells of Arabidopsis. These observations resulted in studies by the present inventors to test whether the heightened presence of APY1 and APY2 in guard cells reflected a role for eATP in guard cell function and whether eATP signaling could be upstream of the NO and ROS signals known to regulate stomatal aperture.

The findings of the present invention indicate the enhanced presence of APY1 and APY2 in guard cells, and reveal that applied ATPγS and ADPβS, which activate eATP responses in plants and animals but are poorly hydrolysable, can induce stomatal opening or closure in a dose-dependent manner. The inventors also show that the effects of applied nucleotides on aperture are accompanied by increases in NO and ROS production, and can be blocked using a purinoceptor inhibitor that blocks eATP responses in animals. These findings are linked to apyrase function by data demonstrating that apyrase expression is dynamically increased when stomates open, and that the chemical inhibition or genetic suppression of apyrases can significantly alter rates of stomatal opening and closing. The results presented herein support the novel and unexpected postulate that eATP is an important factor in guard cell signaling pathways and APY1 and APY2 impact stomatal opening and closure consistent with their hypothesized regulation of eATP.

RT-PCR Analysis of APY Transcripts in Guard Cell Protoplasts: The tissue source for isolating guard cell protoplasts was rosette leaves from Arabidopsis Col-0 and WS ecotypes grown in continuous light for 3 weeks. The isolation procedure used, which yields a protoplast preparation enriched in guard cells, was the overnight method previously described, excluding Histopaque purification steps (Pandey et al., 2002). Total RNA was isolated from the enriched guard cell protoplasts (>50% guard cells) and from whole leaves using the Sigma Spectrum™ Plant Total RNA Kit, following the manufacturer's protocol. Two μg RNA was treated with DNase (Invitrogen) and first-strand cDNA was synthesized with SuperScript III® Reverse Transcriptase (Invitrogen), using the manufacturer's protocol. APY1 and APY2 transcripts were amplified by adding 2 μL, of first-strand cDNA as a template in 25-cycle PCR reactions. For APY1 (At3g04080), the primers AraF172 (5′-GCAGCCGTAACTTGCAATC-3′) (SEQ ID NO: 1) and AAR566 (5′-CACAGCGTAATTCTTCGGACC-3′) (SEQ ID NO: 2) were used, and for APY2 (At5g18280), the primers Arapy2F (5′-GCTTTCCCAAATTCACCGT-3′) (SEQ ID NO: 3) and AAR566 (5′-CACAGCGTAATTCTTCGGACC-3′) (SEQ ID NO: 4) were used (Wu et al. 2007). For ACT2 (At3g18780), the primers 5′-AACTCTCCCGCTATGTATGTCGC-3′ (SEQ ID NO: 5) and 5′-CCATCTCCTGCTCGTAGTCAACA-3′ (SEQ ID NO: 6) were used. The PCR products were run on a 1% agarose gel and visualized under UV light.

Immunoblot Analysis of APY Proteins Extracted from Guard Cell Protoplasts: Guard cell protoplasts and whole leaf tissues were snap frozen with liquid nitrogen, and the whole leaves were ground while frozen. Samples were boiled in a protein extraction buffer containing 0.1 M Tris (pH 6.8), 20% (v/v) glycerol, 5% (w/v) SDS, 200 mM DTT, 200 μM PMSF, and SigmaFAST™ Protease Inhibitor Cocktail Tablets for 3-4 min, and were then centrifuged at 17,000 g for 2 min. The pellet was discarded. The protein concentration of the supernatant was determined using the Bradford assay (Bio-Rad), then 16 μg of this protein was loaded in each lane, separated by SDS-PAGE, and transferred to nitrocellulose. To detect APY1 and APY2, the nitrocellulose was probed with polyclonal guinea pig anti-AtAPY1 antibody (GP1318) (Wu et al., 2007) in a 1:1000 dilution and polyclonal anti-guinea pig IgG antibody coupled to IRDye in a 1:5000 dilution (Rockland) and assayed with the Odyssey infrared imaging system (Li-Cor Biosciences). For Arabidopsis α-tubulin detection with the Odyssey system, monoclonal mouse anti-α-tubulin from sea urchin (1:2500 dilution, Sigma) and polyclonal anti-mouse IgG antibodies coupled to IRDye (1:5000 dilution, Rockland) were used. For immunoblot analyses of the effects of light treatments on APY1 and APY2 protein levels, guard cell protoplasts were dark-adapted for 1 h on ice and equilibrated to room temperature for 10 min. Then the guard cell protoplast preparation was separated into five 100 μL aliquots in 1.5 mL centrifuge tubes, and samples were either untreated (dark control) or treated for 15, 30, 45 and 60 min. Protoplast samples were boiled in extraction buffer and used for immunoblot analyses as described above.

Promoter-GUS Analyses: APY1:GUS and APY2:GUS fusion lines (described in Steinebrunner et al., 2003; Wu et al., 2007), both in the Wassilewskija background, were grown in short days (8 h light; 150 μmol photons m⁻² s⁻¹) at 20° C. (night) and 23° C. (day), respectively. The light intensity was measured with the quantum sensor LI-190SA (LI-COR Biosciences). High humidity conditions (85% relative air humidity (RH)) were achieved by growing the plants covered. Low humidity (33% RH) represented the default condition in the plant growth chamber BrightBoy XL (CFL Plant Climatics, Wertingen, Germany). The RH was determined with a Lutron humidity meter (model HT-315). Two independent lines were used per GUS fusion construct. Plants were grown on well-watered soil for 25 to 42 days. Two rosette leaves per line from two different plants were harvested for each time point. The leaves were fixed in ice-cold 90% (v/v) acetone for 1 h at −20° C., washed three times with 50 mM sodium phosphate (pH 6.8) and stained overnight at 37° C. in staining solution (50 mM sodium phosphate (pH 6.8), 20 mM K₄Fe(CN)₆, 20 mM K₃Fe(CN)₆, 0.2% (v/v) Triton X-100, 1 mM 5-bromo-4-chloro-3-indolyl (3-D glucuronic acid). After the staining procedure, the staining solution was removed and replaced by 70% (v/v) ethanol. Stomates on the abaxial side of the leaves were photographed. The study was performed three times with independent plant cultures.

Stomatal Aperture Studies: Arabidopsis thaliana ecotypes Columbia (Col-0) and Wassilewskija (WS) were used as wild types in this study. Col-0, nia1nia2, atrbohD/F, R2-4A and WS plants were all grown on autoclaved Metro-Mix 200 soil at 22° C. under continuous light. The mutant seeds nia1nia2 nia2-5; CS 2356; Col-0 background) were obtained from Dr. N. M. Crawford (University of California at San Diego) and the mutant seeds atrbohD/F (Col-0 background) were obtained from Dr. J. M. Kwak (University of Maryland). The RNAi mutant apyrase line (R2-4A) is in WS background, so WS ecotype was used as the control wild type for these studies. Leaves from 2 to 3 week old Col-0 plants, or R2-4A and WS plants treated with 4 μM estradiol for 1 week after development of mature basal leaves were used for peel and whole leaf studies. Plants were placed in the dark and watered 24 h before an study. For opening studies, plants were used immediately after 24 h dark treatment and all treatments were done in the dark except for the light treated leaves or peels, thus the control stomata are dark controls. For closure studies, plants were placed in the light 3 h before an study to induce stomatal opening and all treatments were done in the light, thus the control stomata are light controls. For peel studies, epidermal peels were made from the underside of the leaf and treatments were applied to the peels for 1 h. For whole leaf studies, leaves were removed and treatments were applied for 2 h. For each treatment, about 3 or 4 peels collected from different leaves or 3 or 4 different leaves of at least two different plants were floated with the abaxial side up in petri dishes on 3 mL of Arabidopsis leaf buffer consisting of 10 mM KCl, 25 mM MES pH 6.15 (Melotto et al., 2006) and the chemical being tested. For whole leaf studies peels were collected after the treatment.

For studies testing the effects of ATPγS, ADPγS, AMPS, RB2, and PPADS (Sigma, St. Louis, Mo., USA) 20 mM stocks were made by dissolving the compounds in de-ionized water. For studies testing the effects of apyrase inhibitors, 7.5 mg/mL of NGXT191 and of inhibitor #13 (Windsor et al., 2002) was dissolved in dimethyl sulfoxide (DMSO) and then applied at a dilution of 1:000 for a final concentration of 0.1% DMSO. ABA (Sigma, St. Louis, Mo., USA) was dissolved in ethanol in 10 mM stocks and then applied at a dilution of 1:1000. The stocks were stored at −20° C. while not in use. The production of the anti-AtAPY1 antibodies used is described by Steinebrunner et al. (2003). The crude immune and pre-immune sera were purified using protein A-Sepharose following the protocol described by Martin (1982) with the slight modification that the buffers used were azide-free. The protein-A purified sera were both used at a 1:1000 dilution in leaf stomatal aperture studies. The concentration of the immune and pre-immune sera, determined by Bradford assay (Bio-Rad), was 10.2 μg/mL and 7.7 μg/mL, respectively.

After treatment, photos of stomata were taken using a light microscope at 20×. Stomatal aperture width was measured using the image processing software ImageJ. Typically, photos were taken of 70 to 90 stomata per treatment of which only 50 stomata were measured for each treatment. For each treatment the ratio of closed stomata (width of 0) to open stomata was determined for all stomata imaged and this ratio was maintained in the 50 stomata that were measured for each treatment. Data shown uses apertures determined as width/length, however data in all studies also obtained as width only and generally width/length and width only data are in agreement with each other. Statistical significance of the measurements for the treatments was determined using the Student's t test in Microsoft Excel.

Detection and Quantification of H₂O₂ and NO: Col-0 plants were grown on soil for 2 to 4 weeks under continuous light at 21° C. Plants were placed in the dark for 24 h to ensure closure and then transferred to the light for 1 h. After 1 h in the light, mature basal leaves were excised and blended in a Waring blender on the low setting for approximately 10 s to isolate epidermal tissues, and the tissues were placed in 3 mL of 30 mM KCl, 10 mM Mes-KOH, pH 6.16 buffer in light for 2 h (Pei et al., 2000; Murata et al., 2001). DAF-2 DA and H₂DCFDA were dissolved in DMSO to produce 5 mM and 10 mM stock solutions, respectively, and these were stored at −20° C. in 30 μL, aliquots. Fifty μM H₂DCFDA or 15 μM DAF-2 DA was added to the medium in the dark, and after 30 min, 10 μM ABA, 200 μM ATPγS, or buffer (ATP control) was also added to the medium in the dark for 30 min. Peels were then rinsed by decanting the treatment solution and adding 3 mL fresh leaf buffer to the peels. Peels were rinsed twice as described. A peel from each of the three treatments (ABA, ATPγS and buffer) were placed on the same microscope slide and observed sequentially. A second study was staggered 30 to 45 min after the first by loading the H₂DCFDA or DAF-2 DA dye to new peels 30 to 45 min after the first round of dye was added. Confocal laser scanning microscopy (CLSM) was performed with a Leica SP2 AOBS confocal microscope (Leica Microsystems, Bannockburn, Ill., USA). Laser power was set at 15%, with an excitation of 488 nm and an emission of 525 nm. A series of 0.5 μm optical sections with average intensity projection along the z axis were collected and made into one 2D image with greater focal depth. All images were obtained with the same software scanning settings including detector gain and laser intensity settings.

Ecto-luciferase Construct and Plant Transformation: The nucleotide sequence for the 24 amino acid, cleavable signal peptide from the Brassica pollen coat protein, S-locus cysteine-rich protein (SCR13; AF195626) was used to target luciferase for secretion (Schopfer et al., 1999). The signal peptide was incorporated at the N-terminus of the luciferase gene by PCR and the SCR13 signal peptide modified luciferase PCR product was then ligated into the binary vector pLBJ21. This construct was then transformed into Agrobacterium tumefaciens strain GV3101 (pMP90) and then transformed into Arabidopsis plants (Col-0) using the floral dipping method (Clough and Bent, 1998). Transgenic plants were selected by planting on solidified Murashige and Skoog (MS) medium (4.3 g/L Murashige and Skoog salts (Sigma), 0.5% (w/v) MES, and 1.0% (w/v) agar, raised to pH 5.7 with 5 M KOH) containing 50 μg/mL kanamycin. Segregation of T3 generation on kanamycin plates was analysed in order to obtain homozygous lines. Plants showing kanamycin resistance were checked for luciferase activity by growing transgenic plants in MS medium for 10 days and then transferring the whole seedlings into test tubes. Luminescence measurements were performed by placing the test tubes in the light-tight housing of the luminometer reader (Dynatech). Experimental solutions with luciferin substrate with and without 1 mM ATP were injected by an automatic injector, and after 3 seconds counting started at 0.2 second time intervals. Two different transgenic lines x-luc1 and x-luc9 were chosen for this study based on their positive signal without added ATP and an increased signal with 1 mM ATP added and the immediate luminescence of these lines compared to endo-luciferase lines which showed a delay in luminescence.

Imaging of Luminescence in Ecto-luciferase Plants: Ecto-luciferase seeds were surface-sterilized, stratified in the dark in 4° C. for at least three days and then planted directly on a cellophane membrane placed upon solidified MS with 1.0% (w/v) agar. Planted plates were placed upright in a culture chamber and grown at 23° C. under 24-h fluorescent light for 7 days. Plates were then reoriented so that the solidified MS medium was at the bottom of the plate and the seedling was able to grow up into the empty space of the petri dish. Seedlings were then allowed to mature for up to 4 weeks under identical temperature and light settings. For “dark” studies petri dishes of 3-4 week old plants were placed in a dark chamber for 24 hours prior to use in the studies.

Mature basal leaves were excised from x-luc1 and x-luc9 plants 3-4 weeks old. Peels were taken and then immediately floated with the abaxial side up in 40 μL, of Arabidopsis leaf buffer on a microscope slide and the chemical being tested in either light or dark conditions depending on the experimental setup. The placing of the peels within their respective solution was considered time zero. Peels were treated for a minimum of 5 minutes before luciferin and flash buffer were added to the solution. At specified times, 40 μL of luciferin solution (20× stock of D-luciferin, Promega, cat #E160A) diluted to a final concentration of 5 mM in flash assay buffer (20 mM Tricine, 2.67 mM MgSO₄, 0.1 mM EDTA, 2 mM dithiothreitol) as described by Kim et al. (2006) and was added to the existing peel solution in low light conditions, bringing the final concentration of luciferin to 2.5 mM. After placing a coverslip over the sample, imaging was performed immediately using a Leica DME microscope with a Leica HC PLAN APO 20x/0.7 n.a. or HI PLAN 40X/0.65 n.a. objectives, installed in a NightOwl II LB 983 instrument (Berthold Technologies). Luminescence was integrated over 120 seconds, High Gain, Read Out set to slow, using 4×4 pixel binning with Cosmic Suppression on, Background Correction off, and Flatfield Correction off with the filter set to Photo.

All luminescence analysis was conducted using the indiGO Analysis Software (version 2.0.0.26). All images consisted of a photographic image in grayscale with an overlaid luminescence image in pseudo color. Areas of interest were defined manually using the Manual Areas command. This allowed the inventors to manually define integration areas using free form selection of guard cells only. This was accomplished by first lowering the luminescence overlay slider to 0%. This completely removed all luminescence signal from the photographic image. Then the intensity scale of the photographic image was adjusted to improve contrast of the image so that guard cells could be confidently identified from the surrounding pavement cells. Next, the inventors used the Manual Areas command to select only guard cells as areas of interest for luminescence signal integration. Once all areas of interest were manually selected, the inventors brought up the luminescence overlay slider back up to 65%. Finally, the inventors adjusted the intensity scale for the luminescence signal until all background level luminescence was displayed as a dark magenta color, while luminescence levels above background levels were displayed as blue, green, yellow, orange, and red, where red represented the highest level of relative intensity. It is important to note that while adjusting the intensity scale may change the visual representation of measured luminescence they do not change the raw levels of luminescence present, which are used to calculate actual luminescence activity. After all areas of interest had been selected and adjusted, the excel export function was used to create a complete measurement report, including all images and analysis data. All luminescence activity was expressed as average counts per second (cps).

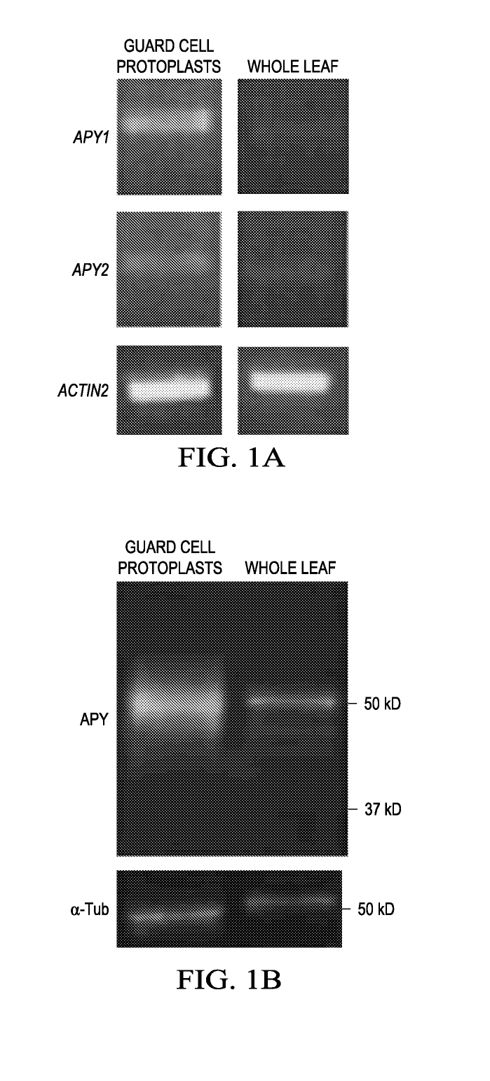

Expression of APY1 and APY2 in Guard Cells: To determine if APY1 and APY2 are expressed in guard cells a RT-PCR analyses of guard cell protoplasts and whole leaf extracts using gene-specific primers was performed. The transcript levels of both APY1 and APY2 are enriched in protoplast preparations in which the ratio of guard cells to mesophyll cells is ≧1.0, compared to whole leaf extracts in which the ratio of guard cells to mesophyll cells is ≦0.1 (FIG. 1A) Immunoblot analyses using polyclonal anti-APY1 antibodies were performed to confirm APY protein expression in protoplast preparations is enriched in guard cells. APY1 and APY2 are 87% identical at the deduced amino acid level and APY1 antibodies have previously been shown to cross-react with both APY1 and APY2 proteins (Wu et al., 2007) Immunoblot results reveal that the cross-reactive band near 50 kDa, the approximate size of APY1 and APY2 proteins (Steinebrunner et al., 2000), is more abundant in the enriched guard cell preparation than in the whole-leaf extracts (FIG. 1B).

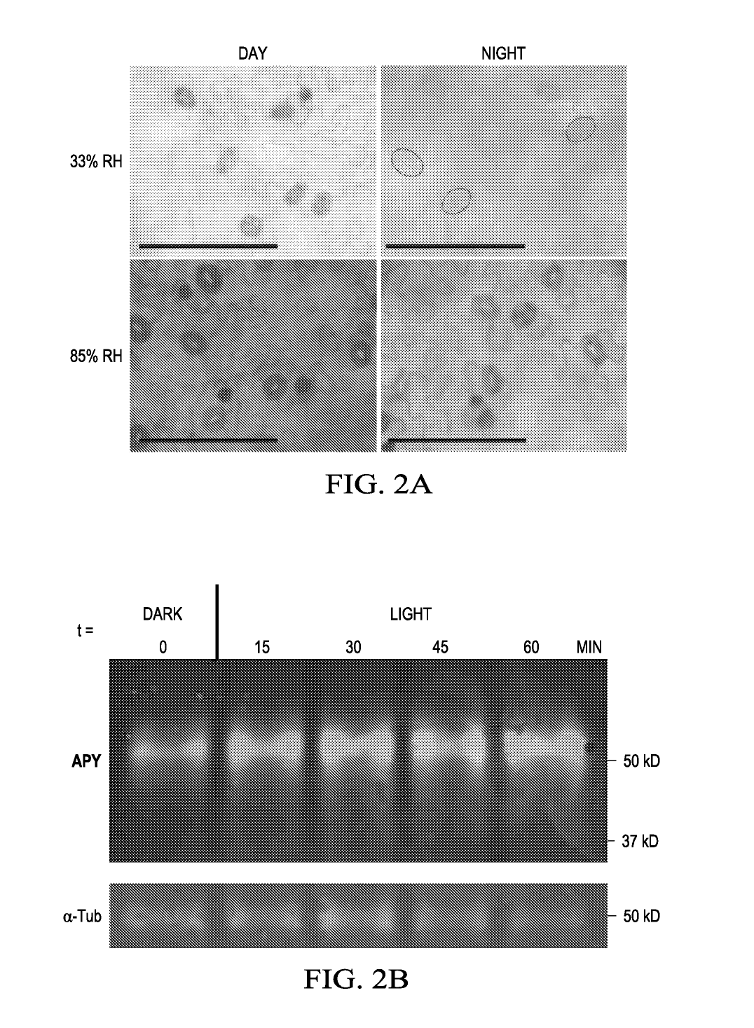

APY1 and APY2 Promoter Activities and Protein Levels Correlate with Open Stomata: To help evaluate whether APY1 and APY2 are involved in the opening and closing of stomates, APY1 and APY2 promoter GUS fusion lines were grown in conditions that either promoted opening or closing of stomata and analysed for GUS activity. During the day, when stomates are generally open, APY1 and APY2 promoter activity was observed in guard cells (FIG. 2A, top left panel) as published previously (Wolf et al., 2007). Higher humidity levels of 85% RH, which increase stomata opening, also increased the GUS staining of the guard cells (FIG. 2A, bottom left panel). On the other hand, closure of stomates in the dark correlated with the decrease of APY1 and APY2 promoter activity (FIG. 2A, top right panel). Under high humidity conditions, stomates will remain open in the dark (Barbour and Buckley, 2007; Mott and Peak, 2010), and, again, guard cells showed high GUS staining (FIG. 2A, bottom right panel). Taken together, APY1 and APY2 promoter activity was high under conditions that induced stomata opening as analysed by GUS staining. In order to determine if the promoter activities had the predicted effects at the protein level and immunoblot analyses of APY1/APY2 protein levels in guard cell protoplasts after treatment with light at various time points was performed. The inventors found that after 15 min of light treatment there was a corresponding increase in level of immunodetectable APY1/APY2 protein and that this increase was maintained over a 1 h period (FIG. 2B).

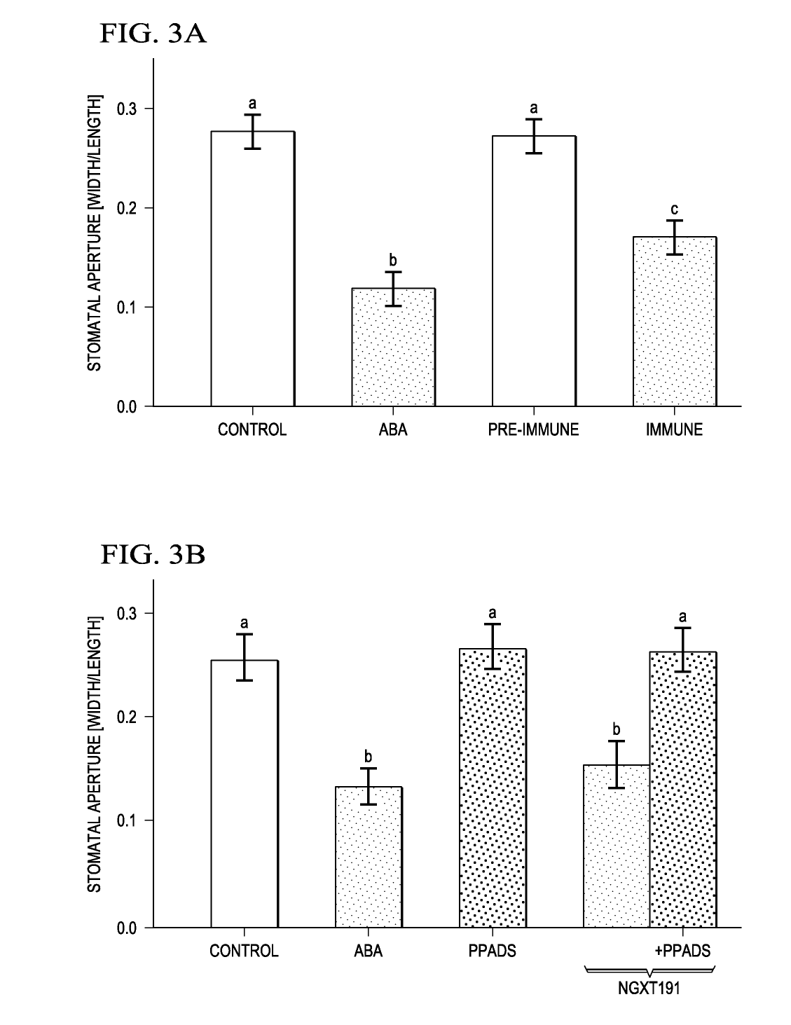

Chemical and Immunological Inhibition of Apyrase Activity Induces Guard Cell Closure: In order to directly determine if apyrase activity plays a role in regulating guard cell aperture in Arabidopsis, the inventors treated epidermal peels and whole leaves with apyrase antibodies and chemical apyrase inhibitors. Anti-APY1 antibodies have previously been shown to inhibit ectoapyrase activity in pollen tubes and cotton fiber cultures (Wu et al., 2007; Clark et al., 2010a), so the inventors tested their effects on stomatal aperture Treatment of epidermal peels and whole leaves with 10 μM ABA and with immune sera induced stomatal closure while treatment with pre-immune sera had no effect on stomatal aperture (FIG. 3A). Chemical apyrase inhibitors have previously been shown to inhibit the activity of APY1 and APY2 (Wu et al., 2007). Treatment of epidermal peels with 7.5 μg/mL of apyrase inhibitor NGXT 191 induced stomatal closure and this closure was blocked by co-incubation with pyridoxalphosphate-6-azo-phenyl-2′,4′-disulphonic acid (PPADS), an antagonist of animal purinoceptors, at a concentration of PPADS (100 μM) that had no effect by itself (FIG. 3B). Treatment of whole leaves with 7.5 μg/mL apyrase inhibitor #13 also caused stomatal closure (data not shown).

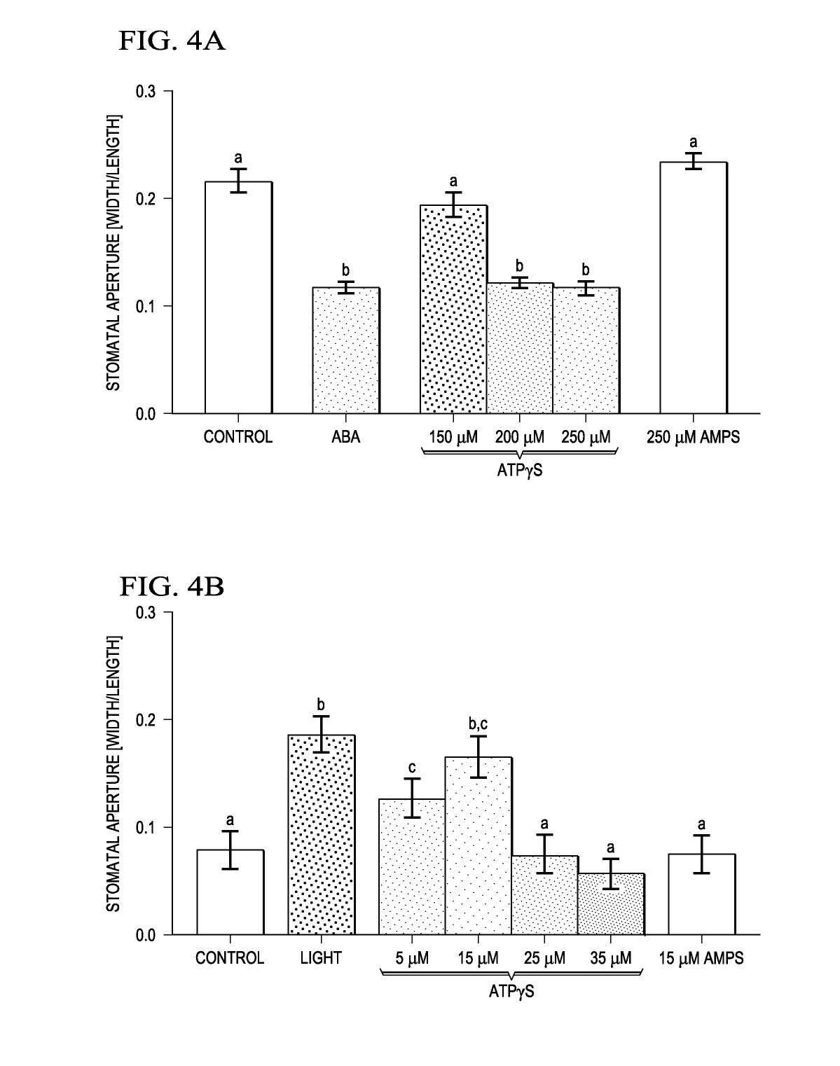

Treatment with ATPγS and ADPβS Induces Changes in Guard Cell Aperture: In order to test whether eATP and eADP might play a role in the regulation of stomatal aperture a dose-response curves for stomatal opening and closure using poorly hydrolysable ATP and ADP analogs, ATPγS and ADPβS was performed. There was a biphasic response to treatment with these nucleotide analogs in epidermal and whole leaf studies: low concentrations of ATPγS induced stomatal opening in darkness and high concentrations of ATPγS induced stomatal closure in the light. In epidermal peel studies, the threshold for ATPγS-induced closure was between 150 and 200 μM, however 250 μM AMPS had no effect on stomatal aperture (FIG. 4A). The inventors found the same threshold for ATPγS-induced closure in leaf studies and for ADPβS-induced closure in peel studies (not shown). The mean stomatal aperture after the closure induced by 200 μM ATPγS was statistically the same as the aperture induced by treatment with 10 μM ABA. In epidermal peel studies, 5 and 15 μM ATPγS induced opening and 15 μM AMPS had no effect on stomatal aperture (FIG. 4B). The mean stomatal aperture induced by low concentrations of ATPγS was smaller than the aperture induced by light treatment. In whole leaf studies, the same biphasic response was observed, and the threshold concentration for closure for both ATPγS and ADPβS was the same as found in the peel studies; however the threshold concentration for opening was shifted higher than 5 μM ATPγS (data not shown).

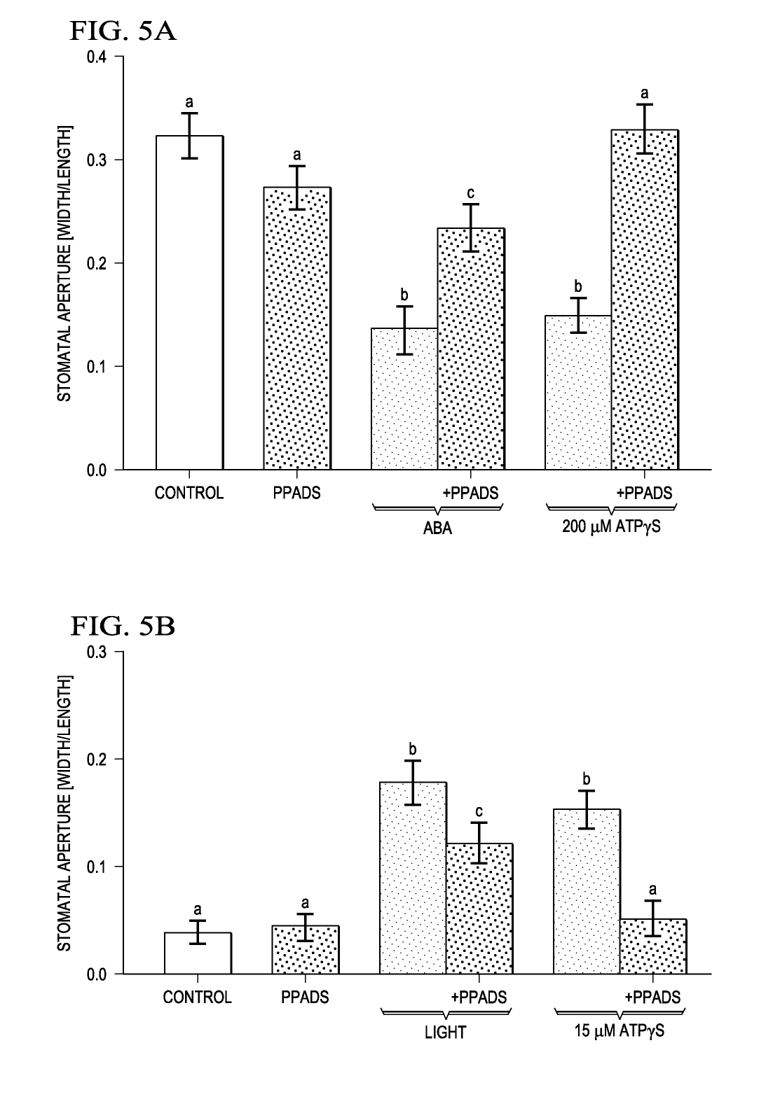

PPADS and RB2 Block the Ability of ATPγS to Regulate Guard Cell Aperture: PPADS and reactive blue 2 (RB2) are well-characterized purinoceptor antagonists that have previously been shown to block ATPγS-induced changes in plant growth responses (Clark et al., 2010a; 2010b). The inventors tested the effects of co-incubation of PPADS and RB2 with different agents on stomatal aperture and found that 100 μM PPADS, which had no effect alone, blocked ATPγS-induced stomatal closure and partially blocked ABA-induced stomatal closure in leaves (FIG. 5A). In epidermal peel studies, PPADS could also block stomatal closure by ATPγS and partially block ABA-induced closure (data not shown). Correspondingly, 100 μM PPADS, which had no effect alone, blocked ATPγS-induced stomatal opening and partially blocked light-induced opening in epidermal peels (FIG. 5B). The inventors observed the same effects of PPADS in opening studies using whole leaves (data not shown). In whole leaf studies RB2 was also able to block ATPγS-induced stomatal closure and partially block ABA-induced stomatal closure in leaves at a concentration (30 μM) that has no effect alone (not shown). RB2 was also able to block ATPγS-induced opening and partially block light-induced opening at a concentration (30 μM) that had no effect by itself in leaves (data not shown).

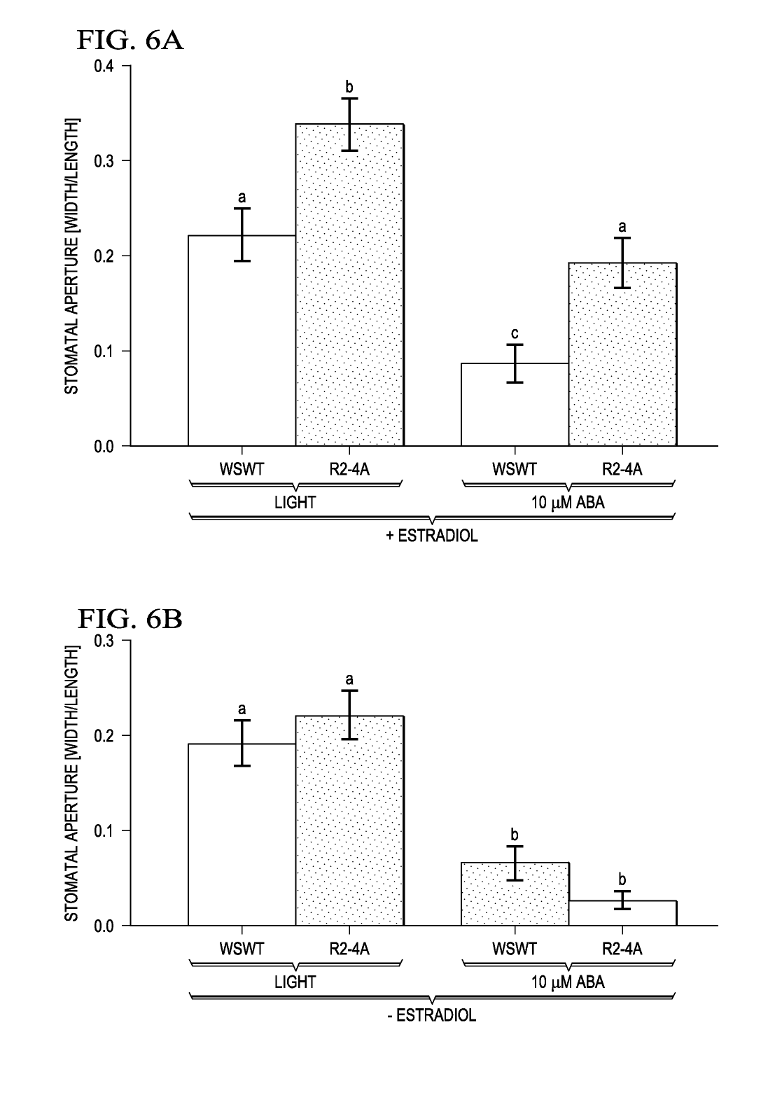

Suppression of Apyrase Expression Affects Regulation of Stomatal Aperture: In order to study the effects of apyrase suppression on stomatal aperture the present inventors used the RNAi line, R2-4A, which is an estradiol-inducible line for the RNAi suppression of APY1 in the background of the APY2 T-DNA knockout line (Wu et al. 2007). After 2 h light treatment, stomata in R2-4A peels were more open than stomata in WS plants (FIG. 6A). In all three biological repeats, the percent of open stomata in R2-4A leaves was 82-93% compared to 68-72% in WS. Even when only open stomata are analyzed, R2-4A stomata were statistically significantly more open than WS stomata after 2 h light treatment. Average stomatal aperture width in estradiol-induced R2-4A mutants ranged from 1.7-3.3 μm while average stomatal aperture width in estradiol-treated WS ranged from 1.0-1.5 μm (data not shown). These control average stomatal widths are low compared to the typical control average stomatal widths for Col-0 in closing studies but are in agreement with average stomatal widths (1.3-3.3 μm) previously reported for closing studies with WS plants (Klein et al., 2003). Treatment with 10 μM ABA caused closure in both estradiol-treated WS wild-type and R2-4A plants, but after 2 h exposure to ABA, stomata in R2-4A peels remained more open than stomata in WS wild-type. In contrast, there were no observable differences in stomatal apertures after treatment with 2 h light or 10 μM ABA when WS wild-type or R2-4A (single apy2 knockout) plants were not treated with estradiol (FIG. 6B).

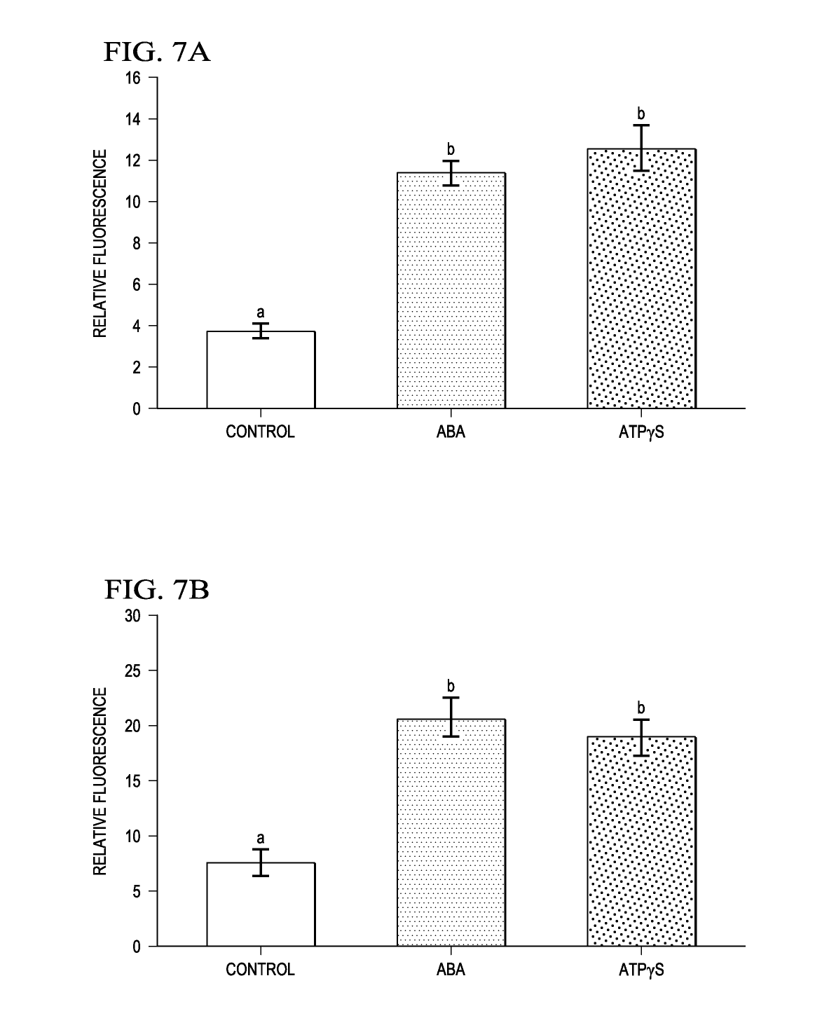

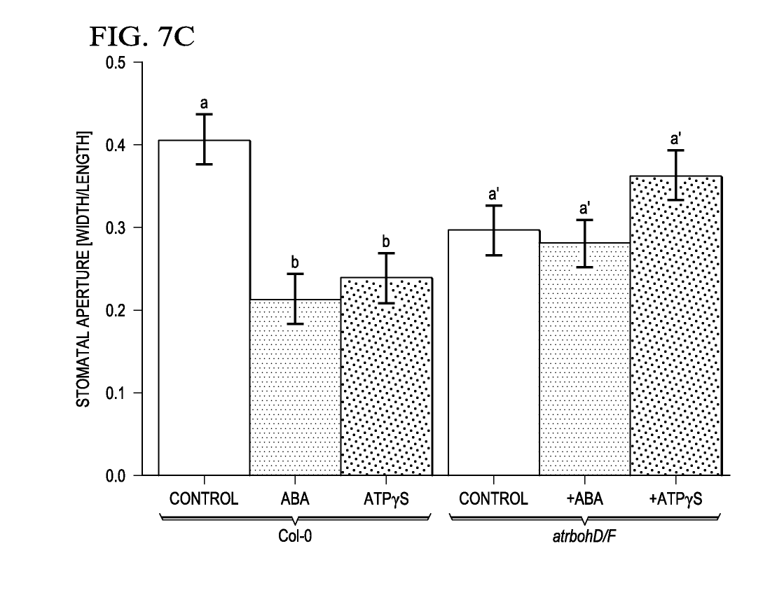

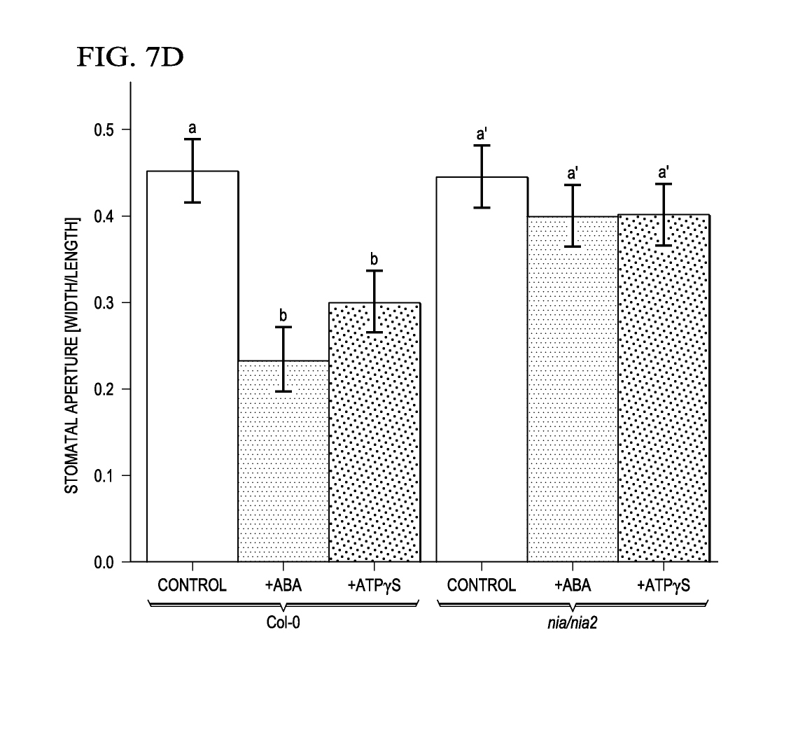

Treatment with 200 μM ATPγS Induces Increased Levels of NO and H₂O₂ in Guard Cells: Treatment of leaf epidermal tissue with 10 μM ABA or 200 μM ATPγS induced a 3-fold increase in H₂DCFDA fluorescence after 30 min in guard cells, but no change in fluorescence in the buffer-treated guard cells (FIG. 7A). Co-incubation with N-acetyl-L-cysteine (NAC), a ROS scavenger, blocked both the ABA- and ATPγS-induced H₂DCFDA fluorescence indicating that this fluorescence is specific for H₂O₂ (data not shown). Treatment of leaf epidermal tissue with 10 μM ABA or 200 μM ATPγS caused a 2.5-fold increase in 2′,7′-dichlorodihydrofluorescein diacetate (DAF-2 DA) fluorescence after 45 min in guard cells, while there was no change in fluorescence observed in the buffer-treated guard cells (FIG. 7B). Co-incubation with cPTIO, an NO scavenger, blocked both the ABA- and ATPγS-induced DAF-2 DA fluorescence indicating that this fluorescence is specific for NO (data not shown).

In order to test the connection between NO and H₂O₂ production and stomatal closure induced by treatment with 200 μM ATPγS the inventors tested the ability of stomata in leaves of nia1nia2 and atrbohD/F mutants to respond to 200 μM ATPγS. The double mutant of nitrate reductase, nia1nia2 has only 0.5% nitrate reductase activity compared to wild-type plants (Wilkinson and Crawford, 1993). The atrbohD/F mutant is disrupted in two subunits of NADPH oxidase that are expressed in guard cells and is deficient in H₂O₂ accumulation in guard cells (Kwak et al., 2003). In closing studies with whole leaves, treatments of atrbohD/F and nia1nia2 mutants with 200 ATPγS or 10 μM ABA had no effect on stomatal aperture (FIGS. 7C and 7D).

Thus, the data presented in FIGS. 7A-7D show that in mutants that cannot make enzymes needed for the synthesis of nitric oxide (nia1nia2 mutants) and of superoxide (rbohD/F mutants), the applied nucleotides cannot regulate stomatal aperture. The results indicate that the induction of NO production and superoxide production by applied nucleotides is required for the nucleotides to regulate stomatal aperture.

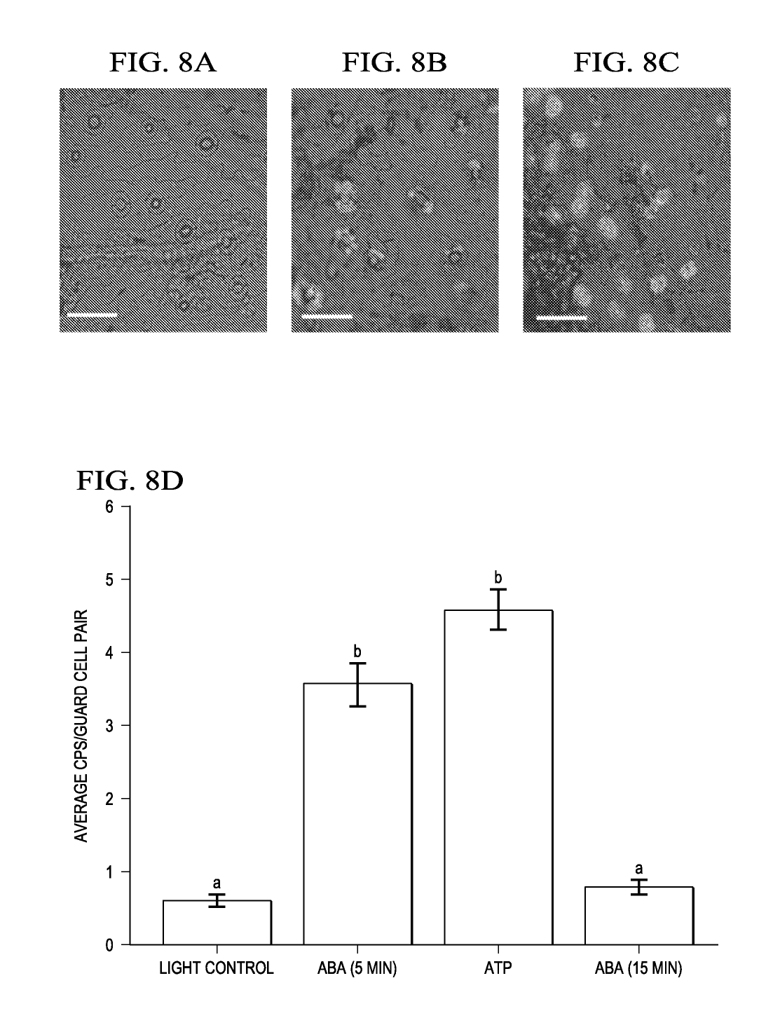

ABA and Light Induce Release of ATP from Guard Cells: In order to monitor the release of ATP from guard cells, transgenic lines expressing a secreted luciferase were generated. To determine if the ecto-luciferase lines could report the presence of eATP in leaves, the inventors first tested the effects of 1 mM ATP on luminescence production. When they were treated with eATP for 5 min or longer, x-luc1 and x-luc9 lines showed high levels of luminescence, on average 4.75 counts per second (cps) (FIGS. 8A and 8B). This luminescence was primarily centered around guard cells, although pavement cells of the epidermis also showed a significant increase in luminescence, on average 1.35 cps, after ATP treatment (FIG. 8C). In trials where no eATP was added and no stimulus was used to cause stomata to change their aperture, only background levels of luminescence were recorded, on average 0.73 cps.

Once the individual plants that were producing luciferase were identified the inventors tested the effect of ABA on epidermal peels of x-luc9 plants that had been exposed to 24 h continuous light and thus had fully open stomata. As early as 5 min after strips were floated on a solution of Leaf Buffer containing 10 μM ABA, luminescent signals were produced in the location of stomata, on average 3.57 cps (FIG. 8B), a level that was statistically higher than control levels. This luminescence signal was maintained for up to 15 min, but after 15 min of continuous exposure to ABA the luminescence signals returned back to control levels, about 0.80 cps (FIG. 8A). The inventors also tested the effects 200 μM ADPβS on open stomata. After 5 min of this treatment, stomata exhibited luminescence levels that averaged 2.75 cps (data not shown), a value that was statistically higher than control levels of luminescence. Quantification of luciferase activity from a representative data set of a closing study is shown in FIG. 8D.

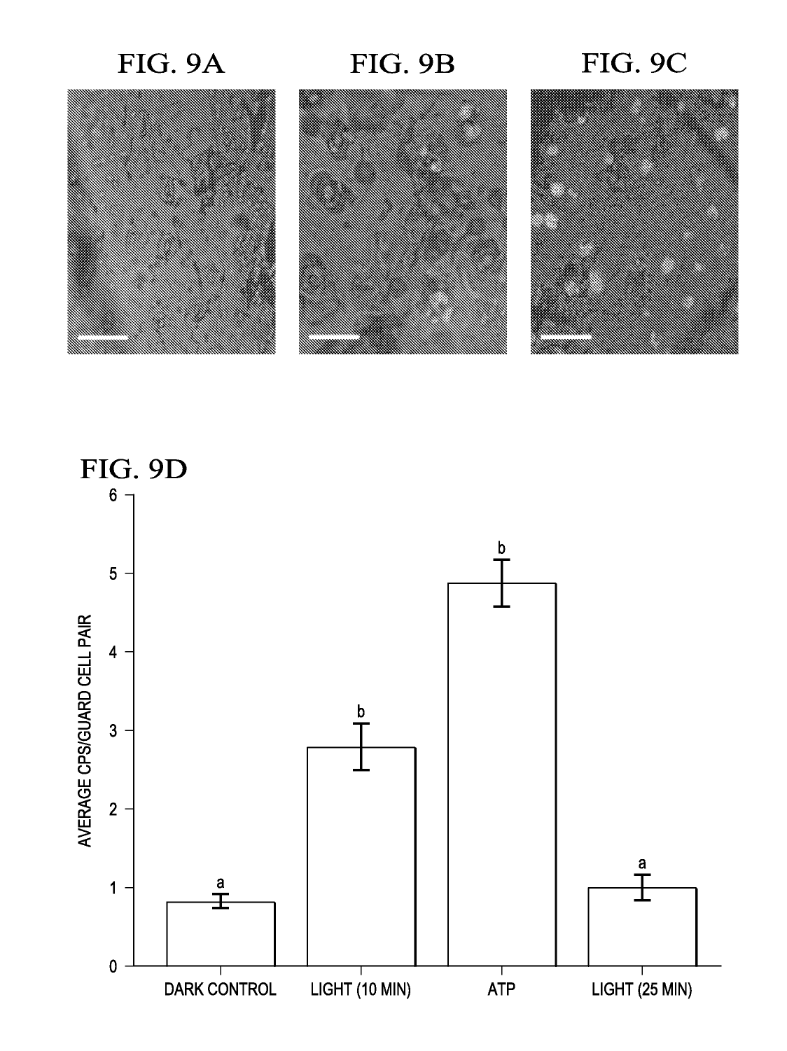

When closed stomata of x-luc9 leaves were exposed to a light stimulus that would cause them to open, the luminescence of stomata in epidermal peels from these leaves increased (FIG. 9B). After the leaves were exposed to 5 min of constant light, and peels from these leaves were floated on Leaf Buffer for 5 additional min in the light, the addition of luciferin plus flash buffer increased stomatal luminescence levels to an average of 2.81 cps, a level statistically higher than the control levels observed after the initial 24 h of darkness (FIG. 9A). Plants were continuously exposed to constant light, and stomatal luminescence levels of peels from their leaves were recorded every 5 min. Luminescence levels peaked after 10 min irradiation and returned back to levels similar to that of control levels (average 1.03 cps) after 25 min and stayed at this low level for up to 35 min of constant light exposure. FIG. 9C shows an epidermal peel from an x-luc9 leaf treated with 1 mM ATP in the dark that shows ecto-luciferase luminescence in guard cells. Quantification of luciferase activity from a representative data set of an opening study is shown in FIG. 9D.

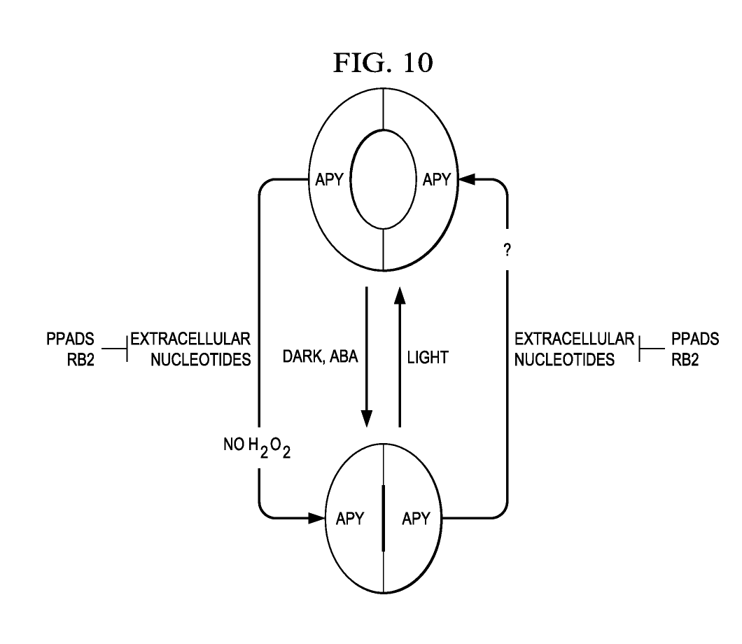

The data obtained using the transgenic plants that express an ecto-luciferase as described hereinabove shows that when guard cells are induced by light to open, or are induced to close by ABA, they release ATP into their extracellular matrix (i.e. into the wall of guard cells). This result supports the model proposed herein and shown in FIG. 10.

Promoter: GUS and RT-PCR analyses indicated that the level of expression of APY1 and APY2 in guard cells was dependent on conditions that control stomatal opening and closing. Higher levels were observed in conditions promoting opening and lower levels in conditions promoting closure, revealing that apyrase expression was linked to guard cell swelling and shrinking Immunoblot results showed that the dynamic increase in APY transcript levels when guard cells open were accompanied by increases in apyrase protein, which occurred rapidly, similar to the dynamic changes in apyrase protein levels that occur during rapid growth changes in hypocotyl cells (Wu et al., 2007). Osmotic swelling of cells induces the release of ATP into the medium (Deter et al., 2004), so the increased expression of APY1 and APY2 in swelling guard cells may be linked to the appearance of increased concentrations of eATP in the extracellular matrix. Increased eATP during cell expansion is correlated with increased APY1 and APY2 expression during the growth of cells and tissues (Kim et al., 2006; Wu et al., 2007). To the extent that the increase in APY in expanding guard cells is driven by changes in [eATP], it would be the consequence rather than the cause of guard cell swelling, nonetheless, these results suggest that there could be a role for these enzymes in regulating stomatal aperture.

Dark-adapted guard cell protoplasts expand when they are exposed to light (Zeiger and Hepler, 1977). When guard cells swell or shrink, the surface area of their plasma membrane changes to accommodate the fluctuating cell volume (Shope et al., 2003), and these volume changes require membrane trafficking (Shope and Mott 2006; Meckel et al. 2007). Exocytosis and stretching of the plasma membrane were previously shown to promote ATP release from cells (Deter et al., 2004; Kim et al., 2006; Weerasinghe et al., 2009), and similar events in expanding guard cells could be the changes that promote ATP release from these cells during stomatal opening. Because APY1 and APY2 are NTPDases, their increased expression when the [eATP] rises suggests that a role for these enzymes is to limit the [eATP]. In Arabidopsis, APY1 and APY2 are reported to function in part as ectoapyrases because polyclonal antibodies that inhibit their activity transiently increase the [eATP] that accumulate in the medium of growing pollen tubes (Wu et al., 2007). However, as pointed out by Wu et al. (2007), APY1 and APY2 could influence [eATP] by their activity in the lumen of Golgi and/or by their activity on the outer face of the plasma membrane.

In both plants and animals ATP release also occurs during hypotonic shock and cell volume decrease (Light et al., 1999; Blum et al., 2010). If this occurs when guard cells shrink in darkness or after ABA treatment, then this release of ATP is not linked to an increase in APY 1 and APY2, because these protein levels are lower in dark-adapted guard cell protoplasts, as are the levels of the transcripts that encode them (FIG. 2A). The combination of ATP release and a decrease in ectoapyrase levels would result in a higher [eATP] during stomatal closure than during its opening, since ectoapyrase levels increase during stomatal opening. Measuring changes in eATP levels during the swelling and shrinking of guard cell protoplasts would be somewhat problematic, for any breakage of the protoplasts during the incubation periods would increase the background [eATP] in the medium and reduce the signal-to-noise ratio. A method for dynamically assaying changes in eATP levels in the ECM around guard cells in intact leaves would have to be developed to solve this problem (Roux et al., 2008).

In order to address the issue whether intact guard cells release ATP on expansion or on shrinkage the present inventors used transgenic lines expressing a luciferase modified to include a signal peptide that would direct it to be secreted. Results using these lines indicated that guard cells do release ATP both when stomata are induced by light to open and when they are induced by ABA to close.

The technique of engineering the secretion of cytoplasmic proteins by attaching a signal peptide has been successfully employed in many reports (Schnell et al., 2010). The signal peptide used to direct the secretion of luciferase into the ECM was that of SCR from Brassica, which definitely promotes the secretion of SCR in pollen (Schopfer et al., 1999; Watanabe et al., 2000). Evidence that it also promotes the secretion of luciferase is that the addition of ATP and luciferin to the incubation medium of x-luc lines 1 and 9 results in a strong luminescence signal that peaks in 3 seconds and then, as the applied ATP is hydrolyzed, goes down to baseline levels within 10 sec, even though some of the luciferin has entered the cell during this time (data not shown). This indicates there is very little luciferase in the cytoplasmic compartment of the x-luc lines to report the cytoplasmic [ATP]. In contrast, in endo-luciferase-expressing lines, even without an addition of ATP to the medium, the luminescence rises sharply as the luciferin enters the cell and continues to rise during a one-minute recording period (data not shown), indicating that the luciferin-luciferase in the cytoplasm is reporting the internal ATP, which remains at non-limiting levels during the recording period. In FIGS. 8C and 9C the applied 1 mM ATP would not be expected to readily cross the plasma membrane, and, even if it did, it would not significantly increase the ATP concentration of the cytoplasm, which is typically near or above mM (Gout et al., 1992).

In an earlier use of x-luc9 plants to assay the pattern of ATP release into the ECM of apical root regions, Roux et al. (2008) reported high [eATP] at the tip and in the elongation zone). This pattern is the same as observed by Kim et al. (2006) using the cellulose-binding-domain-luciferase hybrid protein, which was demonstrated to report [eATP] in Arabidopsis roots.

Applied ATP increases the luminescence of both guard cells and the surrounding pavement cells of the epidermis in peels of x-luc leaves, but the signal in the guard cells is two to three times higher than that in the other epidermal cells. Since the cuticle layer covers all epidermal cells, there is no reason to believe this differential luminescence is due to a more rapid penetration of luciferin into the guard cells. Rather, a more likely explanation is that there is relatively more secretory activity in guard cells (and thus higher ecto-luciferase levels) than in mature pavement cells of the epidermis. This conclusion would be consistent with the fact that there is significantly more membrane turnover in guard cells as they swell and shrink than would be expected in the mature, non-growing pavement cells.

In principle, the lack of luciferase luminescence in stomata that are in a stable open state in light or a stable closed state in darkness (FIGS. 8A and 9A) could be due to a lack of expression of ecto-luciferase or to too low a level of eATP in these cells. However, the fact that applied ATP induces a strong luminescence in these cells demonstrates that the level of available luciferase is not limiting, and thus favors the interpretation that it is the [eATP] that is limiting. The increase in guard-cell pair luminescence after ABA or light treatment, then, is most likely due to an increase in [eATP] induced by these stimuli.

The increase in luciferase luminescence after ABA treatment appears to be significantly greater than after light treatment. As discussed hereinabove, this would be expected if APY levels do not increase when stomates are induced to close, but do increase when they are induced to open. This result would be consistent with the dose-response data that predict higher levels of eATP would induce closing and lower levels would induce opening.

Because APY1 and APY2 are NTPDases, their increased expression when the [eATP] rises suggests that a role for these enzymes is to limit the [eATP]. In Arabidopsis, APY1 and APY2 are reported to function in part as ectoapyrases because polyclonal antibodies that inhibit their activity transiently increase the [eATP] that accumulate in the medium of growing pollen tubes (Wu et al., 2007). However, as pointed out by Wu et al. (2007), APY1 and APY2 could influence [eATP] by their activity in the lumen of Golgi as well as by their activity on the outer face of the plasma membrane.

The inventors directly tested a role for ectoapyrase activity in the control of stomatal closure by treating leaves and peels with chemical inhibitors or anti-APY1/2 antibodies. Both of these treatments induced stomatal closure in the light, similar to the effects of an ABA treatment. Although the chemical inhibitors are small, hydrophobic molecules that could potentially cross the plasma membrane, it is unlikely that the larger antibody molecules could exert their effects inside the cell. These results, then, are consistent with the conclusion that ectoapyrase activity plays an important role in regulating guard cell apertures. The treatments with inhibitors or antibodies were short, 1 h in peel studies and 2 h in leaf studies, and they raised the question of whether genetic suppression of APY1 and APY2 expression over a longer period of time might also affect stomatal apertures.

Total suppression of both APY1 and APY2 expression is lethal (Steinebrunner et al., 2003; Wolf et al., 2007), so partial suppression of these genes by RNAi has been the preferred genetic approach to see how reduction in apyrase expression affects plant growth and development (Wu et al., 2007; Clark et al., 2010b). These RNAi-suppressed plants are APY2 knockouts and are conditionally suppressed in APY1 expression via RNAi under the inducer estradiol (Wu et al. 2007). However, if plants in the RNAi-suppressed line R2-4A are continuously suppressed from germination through flowering, they are dwarf (Wu et al., 2007), and their leaves are too small to fairly compare their stomatal function with wild-type plants. Thus, for the studies to examine stomata function in R2-4A plants, the plants were not treated with estradiol to suppress apyrase expression until after development of the mature basal leaves. This late RNAi induction still suppressed APY1 expression by ˜70%, but it allowed the basal leaves to fully expand before being used in studies.

Although chemical inhibition of apyrase activity and partial suppression of APY expression by RNAi would both reduce ectoapyrase activity, they would not be expected to do so equally. The direct chemical inhibition of the enzyme would likely reduce the ectoapyrase activity significantly more than a partial reduction of the transcript level would. If so, plants treated with the chemical inhibitors would be expected to have a significantly higher [eATP] than the transgenic RNAi-inhibited plants. According to the dose-response assays (FIGS. 4A and 4B), differences in the concentration of applied nucleotides can either promote or inhibit stomata opening.

The dose-response results provide an explanation for why RNAi suppression of apyrase expression leads to increased stomatal aperture in response to light in contrast to the results showing that chemical inhibition of apyrase promotes stomatal closure. That is, the “low” concentration (15 μM) of applied ATPγS that induces opening may be similar to the [eATP] in the apyrase suppressed R2-4A mutant, but the concentration of applied ATPγS that induces closing (≧150 μM) may be similar to the [eATP] established after chemical inhibition of the apyrases. To test this hypothesis a technology for measuring the exact [eATP] in the ECM of plants, which is currently not available, would have to be developed, as noted above.

An alternative explanation for the results observed in RNAi-suppressed plants is that APY1 and APY2 have ectoapyrase functions that affect stomata aperture while they are in the ER and/or Golgi en route to the plasma membrane. Antibody or other chemical suppression of only the ECM-localized ectoapyrase activities of APY1 and APY2 on the plasma membrane would not alter any ER or Golgi function of these apyrases, but genetic suppression of APY1/APY2 would.

The effects of ATPγS on stomatal aperture are likely mediated by a plant eATP receptor that is pharmacologically similar to animal purinoceptors, because PPADS is able to block eATP-induced stomatal closure and opening. This inhibitor also partially blocks the ability of ABA to induce stomatal closure and light to induce stomatal opening, suggesting that eATP may play a critical role in the complex signaling pathway mediated by ABA and light. Although a purinoceptor-like protein has been discovered in algae (Fountain et al., 2008), none has yet been found in higher plants. The discovery and characterization of a receptor for extracellular nucleotides in leafy plants will be a necessary pre-requisite to clarify more completely the role of extracellular nucleotides in controlling guard cell responses to hormonal and environmental cues.

In Arabidopsis the production of NO and ROS is both induced by extracellular nucleotides (Song et al., 2006; Reichler et al., 2009; Clark et al., 2010b; Tonón et al. 2010) and required in the signaling pathway that links applied nucleotides to growth changes (Reichler et al., 2009; Clark et al., 2010b). The induction of these signaling intermediates by eATP in other species is also well established (Kim et al., 2006; Foresi et al., 2007; Wu et al., 2008; Clark et al., 2010b; Terrile et al., 2010). Because H₂O₂ and NO play roles in stomatal closure in response to ABA and stressors (Garcia-Mata and Lamattina, 2001; Desikan et al., 2002; Kwak et al., 2003; Neill et al., 2008), the inventors tested the effects of applied nucleotides on their production in guard cells, and found that concentrations of ATPγS that induced closure also increased levels of H₂O₂ and NO in guard cells to near levels induced by a concentration of ABA that induces stomatal closure. Interestingly, ATPγS-induced H₂O₂ production appeared to occur faster than ATPγS-induced NO production in guard cells, consistent with an earlier observation that ABA-induced NO production in guard cells is dependent on H₂O₂ synthesis (Bright et al., 2006).

The production of NO is not absolutely required for ABA to induce stomatal closure in all circumstances (Ribeiro et al., 2009; Lozano-Juste and Leon, 2010). For example, NO is not required for ABA-induced closure when plants are experiencing dehydration (Ribeiro et al., 2009; Lozano-Juste and Leon, 2010). In studies described in the present invention, the plants were well hydrated, and the results agree with previous reports that in these conditions nia1nia2 and atrbohD/F mutant stomata are unable to close in response to ABA treatment (Desikan et al., 2002; Kwak et al., 2003; Bright et al., 2006; Hao et al., 2010). The fact that these mutants also do not respond to treatment with 200 μM ATPγS indicates that NO and H₂O₂ help mediate the effects of nucleotides on stomatal closure.

FIG. 10 illustrates some of the main findings of the present invention in a hypothetical model. The model shows that extracellular nucleotides can regulate both the opening and closing of stomata, with the closing response requiring a higher concentration of nucleotides. It shows that the mammalian purinoceptor antagonists PPADS and RB2 inhibit the ATPγS- or ADPγS-induced stomatal aperture changes, which suggests that the extracellular nucleotides are recognized by purinergic-like receptors. The model predicts that NO and H₂O₂ changes already documented to induce stomatal closure (Mata and Lamattina, 2001; Bright et al., 2006; Desikan et al., 2006) are downstream of increases in extracellular nucleotides. In principle, NO and H₂O₂ production could also be induced by the ATP released during guard cell swelling. In this regard it is to be noted that NO and H₂O₂ help mediate both the stimulation and the inhibition of root hair growth by applied nucleotides. The model indicates that the light and dark treatments that alter the volume of guard cells also alter their content of apyrase transcripts and protein.

In animal cells hypotonic stress and cell shrinkage is accompanied by ATP release (Light et al., 1999; Blum et al., 2010), just as happens during hypertonic stress and cell swelling. The model presented hereinabove predicts that both the swelling and shrinking of guard cells would induce a release of ATP, but because the shrinking response is accompanied by a decrease in ectoapyrase content and the swelling is accompanied by an increase in ectoapyrase content, the equilibrium [eATP] would be higher during the closing response.

The data presented herein strongly favour the conclusion that APY1 and APY2 and extracellular nucleotides play key roles in the control of stomatal aperture and changes in APY and extracellular nucleotide can lead to better-characterized hormonal and environmental cues to control guard cell function.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

1. U.S. Patent Publication No. 20080274888: N-Acetylcysteine Amide (Nac Amide) for Enhancing Plant Resistance and Tolerance to Environmental Stress.
2. U.S. Patent Publication No. 20090328246: Use of the OCP3 Mutation to Regulate Drought Resistance in Plants.
3. U.S. Patent Publication No. 20080254986: Use of Piperonyl Butoxide to Protect Plants.
4. WIPO Patent Application No. WO/2010/065725: Regulation of Cotton Fiber Growth by Extracellular Nucleotides and Ectoapyrases.
5. Barbour M M, Buckley T N (2007) The stomatal response to evaporative demand persists at night in Ricinus communis plants with high nocturnal conductance. Plant Cell Environ 30: 711-721.
6. Blum A E, Walsh B C, Dubyak G R (2010) Extracellular osmolarity modulates G protein-coupled receptor-dependent ATP release from 1321N1 astrocytoma cells. Amer J Physiol-Cell Physiol 298: C386-C396.
7. Bright J, Desikan R, Hancock J, Weir I, Neill S (2006) ABA-induced NO generation and stomatal closure in Arabidopsis are dependent on H₂O₂ synthesis. Plant J 45: 113-122.
8. Burnstock G (2008) Purinergic signalling and disorders of the central nervous system. Nat Rev Drug Discovery 7: 575-590.
9. Clark G, Roux S J (2009) Extracellular nucleotides: ancient signaling molecules. Plant Sci 177: 239-244.
10. Clark G, Torres J, Handley C, Guan X Y, Lee J L, Kays J E, Finlayson S, Chen, Z J, Roux S J (2010a) Apyrase (Nucleoside triphosphate-diphosphohydrolase) and extracellular nucleotides regulate cotton fiber elongation in cultured ovules. Plant Physiol 152: 1073-1083.
11. Clark G, Wu M, Wat N, Onyirimba J, Pham T, Herz N, Ogoti J, Gomez D, Canales A A, Aranda G, Blizard M, Nyberg T, Terry A, Torres J, Wu J, Roux S J (2010b) Both the stimulation and inhibition of root hair growth induced by extracellular nucleotides in Arabidopsis are mediated by nitric oxide and reactive oxygen species. Plant Mol Biol 74: 423-435.
12. Clough S J, Bent A F (1998) Floral dip: a simplified method for Agrobacterium-mediated transformation of Arabidopsis thaliana. Plant J 16: 735-743.
13. Demidchik V, Nichols C, Oliynyk M, Dark A, Glover B, Davies J (2003) Is ATP a signaling agent in plants? Plant Physiol 133: 456-461.
14. Demidchik V, Shang Z, Shin R, Thompson E, Rubio L, Laohavisit A, Mortimer J, Chivasa S, Slabas A, Glover B, Schachtman D, Shabala S, Davies J (2009) Plant extracellular ATP signaling by plasma membrane NADPH oxidase and Ca²⁺ channels. Plant J 58: 903-913.
15. Desikan R, Griffiths R, Hancock J, Neill S (2002) A new role for an enzyme: nitrate reductase-mediated nitric oxide generation is required for abscisic acid-induced stomatal closure in Arabidopsis thaliana. Proc Natl Acad Sci USA 99: 16314-16318.
16. Desikan R, Last K, Harrett-Williams R, Tagliavia C, Harter K, Hooley R, Hancock J T, Neill S J (2006) Ethylene-induced stomatal closure in Arabidopsis occurs via AtrbohF-mediated hydrogen peroxide synthesis. Plant J 47: 907-916.
17. Foresi N, Laxalt A, Tonón C, Casalongué C, Lamattina L (2007) Extracellular ATP induces nitric oxide production in tomato cell suspensions. Plant Physiol 145: 589-592.
18. Fountain S, Cao L, Young M, North R A (2008) Permeation properties of a P2X receptor in the green algae Ostreococcus tauri. J Biol Chem 283: 15122-15126.
19. Garcia-Mata C, Lamattina L (2001) Nitric oxide induces stomatal closure and enhances the adaptive plant responses against drought stress. Plant Physiol 126: 1196-1204.
20. Gout E, Bligny R, Douce R (1992) Regulation of intracellular pH values in higher plant cells. J Biol Chem 267: 13903-13909.
21. Hao F S, Zhao S L, Dong H, Zhang H, Sun L R, Miao C (2010) Nia1 and Nia2 are involved in exogenous salicylic acid-induced nitric oxide generation and stomata closure in Arabidopsis. J Integ Plant Biol 52: 298-307.
22. Jeter C, Tang W, Henaff E, Butterfield T, Roux S J (2004) Evidence of a novel cell signaling role for extracellular adenosine triphosphates and diphosphates in Arabidopsis. Plant Cell 16: 2652-2664.
23. Kim S-Y, Sivaguru M, Stacey G (2006) Extracellular ATP in plants. Visualization, localization, and analysis of physiological significance in growth and signaling. Plant Physiol 142: 984-992.
24. Klein M, Perfus-Barbeoch L, Frelet A, Gaedeke N, Reinhardt D, Mueller-Roeber B, Martinoia E, Forestier C (2003) The plant multidrug resistance ABC transporter AtMRP5 is involved in guard cell hormonal signalling and water use. Plant J 33: 119-129.
25. Kwak J M, Mori I C, Pei Z M, Leonhardt N, Torres M A, Dangl J L, Bloom R E, Bodde S, Jones J D G, Schroeder J I (2003) NADPH oxidase AtrbohD and AtrbohF genes function in ROS-dependent ABA signaling in Arabidopsis. EMBO J 22: 2623-2633.
26. Light D B, Capes T L, Gronau R T, Adler M R (1999) Extracellular ATP stimulates volume decrease in Necturus red blood cells. Amer J Physiol-Cell Physiol 277: C480-C491.
27. Lozano-Juste J, Leon J (2010) Enhanced abscisic acid-mediated responses in nia1nia2noa1-2 triple mutant impaired in NIA/NR- and AtNOA1-dependent nitric oxide biosynthesis in Arabidopsis. Plant Physiol. 152: 891-903.
28. Mata C G, Lamattina L (2001) Nitric oxide induces stomatal closure and enhances the adaptive plant responses against drought stress. Plant Physiol 126: 1196-1204.
29. Meckel T, Gall L, Semrau S, Homann U, Thiely G (2007) Guard cells elongate: Relationship of volume and surface area during stomatal movement. Biophys J 92: 1072-1080.
30. Martin L N (1982) Separation of guinea pig IgG subclasses by affinity chromatography of protein A-Sepharose. J Immunol Methods 52: 205-212.
31. Melotto M, Underwood W, Koczan J, Nomura K, He S Y (2006) Plant stomata function in innate immunity against bacterial invasion. Cell 126: 969-980.
32. Mott K A, Peak D (2010) Stomatal responses to humidity and temperature in darkness. Plant Cell Environ 33: 1084-1090.
33. Murata Y, Pei Z M, Mori I C, Schroeder J (2001) Abscisic acid activation of plasma membrane Ca²⁺ channels in guard cells requires cytosolic NAD(P)H and is differentially disrupted upstream and downstream of reactive oxygen species production in abi1-1 and abi2-1 protein phosphatase 2C mutants. Plant Cell 13: 2513-2523.
34. Neill S, Banos R, Bright J, Desikan R, Hancock J, Harrison J, Morris P, Ribeiro D, Wilson I (2008) Nitric oxide, stomatal closure, and abiotic stress. J Exp Bot 59: 165-176.
35. Pandey S, Wang X, Coursol S, Assmann S (2002) Preparation and applications of Arabidopsis thaliana guard cell protoplasts. New Phytol 153: 517-526.
36. Pei Z-M, Murata Y, Benning G, Thomine S, Kliisener B, Allen G, Grill E, Schroeder J (2000) Calcium channels activated by hydrogen peroxide mediate abscisic acid signalling in guard cells. Nature 406: 731-734.
37. Reichler S A, Torres J, Rivera A L, Cintolesi V A, Clark G, Roux S J (2009) Intersection of two signaling pathways: Extracellular nucleotides regulate pollen germination and pollen tube growth via nitric oxide. J Exp Bot 60: 2129-2138.
38. Ribeiro D M, Desikan R, Bright J, Confraria A, Harrison J, Hancock J T, Barros R S, Neill S J, Wilson I D (2009) Differential requirement for NO during ABA-induced stomatal closure in turgid and wilted leaves. Plant Cell Environ 32: 46-57.
39. Roux S J, Steinebrunner I (2007) Extracellular ATP: an unexpected role as a signaler in plants. Trends Plant Sci 12: 522-527.
40. Roux S, Wu J, Henaff E, Torres J, Clark G (2008) Regions of growth are regions of highest release of ATP and highest expression of ectonucleotidases AtAPY1 and AtAPY2 in Arabidopsis. Purinergic Signalling 4: S112-S112.
41. Schnell J A, Han S Y, Mild B L, Johnson D A (2010) Soybean peroxidase propeptides are functional signal peptides and increase the yield of a foreign protein. Plant Cell Rep 29: 987-996.
42. Shope J, DeWald D, Mott K (2003) Changes in surface area of intact guard cells are correlated with membrane internalization. Plant Physiol 133: 1314-1321.
43. Shope J C, Mott K A (2006) Membrane trafficking and osmotically induced volume changes in guard cells. J Exp Bot 57: 4123-4131.
44. Schopfer C R, Nasrallah M E, Nasrallah J B (1999) The male determinant of self-incompatibility in Brassica. Science 286: 1697-1700.
45. Song C, Steinebrunner I, Wang X, Stout S, Roux S (2006) Extracellular ATP induces the accumulation of superoxide via NADPH oxidases in Arabidopsis. Plant Physiol 140: 1222-1232.
46. Steinebrunner I, Jeter C, Song C, Roux S J (2000) Molecular and biochemical comparison of two different apyrases from Arabidopsis thaliana. Plant Physiol Biochem 38: 913-922.
47. Steinebrunner I, Wu J, Sun Y, Corbett A, Roux S J (2003) Disruption of apyrases inhibits pollen germination in Arabidopsis. Plant Physiol 131: 1638-1647.
48. Tanaka K, Gilroy S, Jones A M, Stacey G (2010a) Extracellular ATP signaling in plants. Trends Cell Biol 20: 601-608.
49. Tanaka K, Swanson S J, Gilroy S, Stacey G (2010b) Extracellular nucleotides elicit cytosolic free calcium oscillations in Arabidopsis. Plant Physiol 154:705-719.
50. Terrile M C, Tonón CV, Iglesias M J, Lamattina L, Casalongué CA (2010) Extracellular ATP and nitric oxide signaling pathways regulate redox-dependent responses associated to root hair growth in etiolated Arabidopsis seedlings. Plant Signal Behav 5: 1-4.
51. Tonón C, Terrile M C, Iglesias M J, Lamattina L, Casalongué C (2010) Extracellular ATP, nitric oxide and superoxide act coordinately to regulate hypocotyl growth in etiolated Arabidopsis seedlings. J Plant Physiol 167:540-546.
52. Watanabe M, Suzuki G, Takayama S, Isogai A, Hinata K (2000) Genomic organization of the SLG/SRK region of the S locus in Brassica species. Ann Bot 85: 155-160.
53. Weerasinghe R R, Swanson S J, Okada S F, Garrett M B, Kim S Y, Stacey G, Boucher R C, Gilroy S, Jones A M (2009) Touch induces ATP release in Arabidopsis roots that is modulated by the heterotrimeric G-protein complex. FEBS Lett 583: 2521-2526.
54. Windsor J B, Thomas C, Hurley L, Roux S J, Lloyd A M (2002) Automated colorimetric screen for apyrase inhibitors. Biotechniques 33: 1024, 1026, 1028-1030.
55. Wilkinson J Q, Crawford N M (1993) Identification and characterization of a chlorate-resistant mutant of Arabidopsis thaliana with mutations in both nitrate reductase structural genes Nia1 and Nia2. Mol Gen Genet 239: 289-297.
56. Wolf C, Hennig M, Romanovicz, D, Steinebrunner I (2007) Developmental defects and seedling lethality in apyrase AtAPY1 and AtAPY2 double knockout mutants. Plant Mol Biol 64: 657-672.
57. Wu J, Steinebrunner I, Sun Y, Butterfield T, Torres J, Arnold D, Gonzalez A, Jacob F, Reichler S, Roux S J (2007) Apyrases (nucleoside triphosphate-diphosphohydrolases) play a key role in growth control in Arabidopsis. Plant Physiol 144: 961-975.
58. Wu S, Wu J (2008) Extracellular ATP-induced NO production and its dependence on membrane Ca²⁺ flux in Salvia miltiorrhiza hairy roots. J Exp Bot 59: 4007-4016.
59. Zeiger E, Hepler P K (1977) Light and stomatal function—blue-light stimulates swelling of guard cell protoplasts. Science 196: 887-889.

What is claimed is:

1. An agricultural composition for increasing a resistance or a tolerance of a plant to one or more environmental stress conditions comprising at least one of an extracellular exogenous nucleotides selected from di-nucleotides, tri-nucleotides, or poorly-hydrolyzable nucleotides, an ectopyrase activity inhibitor, a modulator of an ectoapyrase gene transcription, an anti-ectoapyrase antibody or fragments thereof at a concentration sufficient to increase the resistance or the tolerance of the plant to the one or more environmental stress conditions.

2. The composition of claim 1, wherein the one or more poorly-hydrolyzable nucleotides comprise thio, methylene, amide or methyl-modified ATPγS, ADPβS, ATP, ADP, UTP, UDP, CTP, CDP, TTP, TDP, GTP, GDP, dATP, dADP, dUTP, dUDP, dCTP, dCDP, dTTP, dTDP, dGTP, dGDP, and analogues and combinations thereof.

3. The composition of claim 1, wherein the one or more poorly-hydrolyzable nucleotides comprise ATPγS, ADPβS, or both.

4. A method for increasing a resistance or a tolerance of a plant to one or more environmental stress conditions comprising the step of administering to the surface of the plant an agricultural composition comprising at least one of an extracellular exogenous nucleotides selected from di-nucleotides, tri-nucleotides, or poorly-hydrolyzable nucleotides, an ectopyrase activity inhibitor, a modulator of an ectoapyrase gene transcription, an anti-ectoapyrase antibody or fragments thereof in an amount effective to induce resistance or tolerance in the plant against the one or more environmental stress conditions.

5. The method of claim 4, wherein the one or more poorly-hydrolyzable nucleotides comprise thio, methylene, amide or methyl-modified ATPγS, ADPβS, ATP, ADP, UTP, UDP, CTP, CDP, TTP, TDP, GTP, GDP, dATP, dADP, dUTP, dUDP, dCTP, dCDP, dTTP, dTDP, dGTP, dGDP, and analogues and combinations thereof.

6. The method of claim 4, wherein the one or more poorly-hydrolyzable nucleotides comprise ATPγS, ADPβS, or both.

7. The method of claim 4, wherein the environmental stress conditions are selected from the group consisting of extreme temperature or weather conditions, drought, frost, rain, hail, moisture, humidity, heat, excess salinity, excess minerals, poor soil nutrients, poor growth medium nutrients, insects, arachnids, nematodes, and other pest infestations, bacteria, fungi, viruses, mycoplasms, and other pathogens, and other biotic or abiotic environmental stress conditions.

8. The method of claim 4, wherein the environmental stress condition comprises drought, pathogens or both.

9. The method of claim 4, wherein the composition is administered to the leaf of the plant.

10. The method of claim 4, wherein the composition is administered by spraying the plant, direct application to the surface of the plant, pouring onto the plant, drenching the root system of the plant, administered as a mixture with soil, water, nutrients, or manure, or any combinations thereof.

11. A composition to modulate an extracellular exogenous nucleotide regulated opening or closing of one or more stomatal pores in a plant comprising at least one of a mammalian purinoreceptor antagonist, wherein the mammalian purinoreceptor antagonist is selected from the group consisting of pyridoxalphosphate-6-azo-phenyl-2′,4′-disulphonic acid (PPADS), Reactive Blue 2 (RB-2), or both.

12. The composition of claim 11, wherein the composition may comprise one or more additional mammalian purinoreceptor antagonists selected from the group consisting of 8′[carbonylbis(imino-3,1-phenylcarbonylimino)]bus-1,3,5-naphthalene-trisulphonic acid (NF023), 2′,3′-O-(2,4,6-trinitrophenyl)-ATP (NT-ATP), pyridoxal-a5-phosphate-6-phenylazo-4′-carboxylic acid (MRS2159), 8,8′-(carbonylbis(imino-4,1-phenylenecarbonyl-imino-4,1-phenylenecarbonylimino))bis(1,3,5-naphthalenetrisulfonic acid) (NF279), 4,4′,4″,4′″-[carbonylbis(imino-5,1,3-benzenetriyl-bis(carbonylimino))]tetrakis-1,3-benzenedisulfonic acid (NF449), brilliant blue G (BB-G), 4′,4″,4′″-[carbonylbis(imino-5,1,3-benzenetriyl bis(carbonylimino))]tetrakisbenzenesulfonic acid (NF110), 1-[N,O-bis(5-Isoquinolinesulfonyl)-N-methyl-L-tyrosyl]-4-phenylpiperazine (KN-62), 3-[[5-(2,3-Dichlorophenyl)-1H-tetrazol-1-yl]methyl]pyridine (A438079), 2′deoxy-N⁶-methyladenosine-3′5′-biphosphate (MRS2179), 2-chloro-N⁶-methyl-(N)-methanocarba-2′-deoxyadenosine3′,5′-biphosphate (MRS2279), 2-iodo-N6-methyl-(N)-methanocarba-2′-deoxyadenosine3′,5′-biphosphate (MRS2500), N,N″-1,4-butanediylbis[N′-(3-isothiocynatophenyl)thio urea (MRS2578), 8,8′-[carbonylbis[imino-3,1-phenylenecarbonylimino(4-fluoro-3,1-phenylene)carbonylimino]]bis-1,3,5-naphthalene trisulfonic acid (NF157), N6-(2-methylthioethyl)-2-(3,3,3-trifluoropropylthio)-β-γ-dichloromethylene-ATP (cangrelor), clopidogrel, 2-[(2-chloro-5-nitrophenyl)azo]-5-hydroxy-6-methyl-3[(phosphonooxymethyl]-4-pyridinecarboxaldehyde (MRS2211), UDP, and any combinations thereof.

13. The composition of claim 11, wherein the mammalian purinoreceptor antagonist blocks a dose-dependent stomatal opening or closure mediated by the extracellular exogenous nucleotides.

14. The composition of claim 11, wherein the mammalian purinoreceptor antagonist blocks a dose-dependent stomatal opening or closure mediated by the extracellular exogenous nucleotides by an inhibition of extracellular ATP (eATP) mediated cell signaling in the plants.

15. A method for modulating an extracellular exogenous nucleotide regulated opening or closing of one or more stomatal pores in a plant comprising the step of administering to a leaf surface of the plant at least one of a mammalian purinoreceptor antagonist, wherein the mammalian purinoreceptor antagonist comprises pyridoxalphosphate-6-azo-phenyl-2′,4′-disulphonic acid (PPADS), Reactive Blue 2 or both in an amount effective to modulate an extracellular exogenous nucleotide regulated opening or closing of the one or more stomatal pores.

16. The method of claim 15, wherein the extracellular exogenous nucleotides are selected from the group consisting of di-nucleotides, tri-nucleotides, or poorly-hydrolyzable nucleotides.

17. The method of claim 16, wherein the poorly-hydrolyzable nucleotides comprises thio, methylene, amide or methyl-modified ATP, ADP, UTP, UDP, CTP, CDP, TTP, TDP, GTP, GDP, dATP, dADP, dUTP, dUDP, dCTP, dCDP, dTTP, dTDP, dGTP, dGDP, ATPγS, ADPβS, and analogues and combinations thereof.

18. The method of claim 15, wherein the mammalian purinoreceptor antagonist blocks a dose-dependent stomatal opening or closure mediated by the extracellular exogenous nucleotides.

19. A composition to reduce water loss, transpiration, wilting or any combinations thereof in a plant due to excessive sunlight, artificial light, heat or any combinations thereof comprising at least one of a mammalian purinoreceptor antagonist, wherein the mammalian purinoreceptor antagonist comprises pyridoxalphosphate-6-azo-phenyl-2′,4′-disulphonic acid (PPADS), Reactive Blue 2 or both, wherein the mammalian purinoreceptor antagonist partially blocks a light induced opening of the one or more stomatal pores.

20. A method of reducing water loss, transpiration, wilting or any combinations thereof in a plant due to excessive sunlight, artificial light, heat or any combinations thereof comprising the step of administering to a leaf surface of the plant at least one of a mammalian purinoreceptor antagonist, wherein the mammalian purinoreceptor antagonist comprises pyridoxalphosphate-6-azo-phenyl-2′,4′-disulphonic acid (PPADS), Reactive Blue 2, (RB-2), or both in an amount effective to reducing water loss, transpiration, wilting or any combinations thereof by partially blocking a light induced opening of the one or more stomatal pores.

21. An agricultural composition for increasing a resistance or a tolerance to drought, pathogens or both comprising at least one of a chemical ectopyrase activity inhibitor, a modulator of an ectoapyrase gene transcription, an anti-ectoapyrase antibody or fragments thereof at a concentration sufficient to increase the resistance or the tolerance of the plant to drought, pathogens or both.

22. The composition of claim 21, wherein the chemical ectopyrase activity inhibitor is selected from the group consisting of N-(3-methylphenyl)-4-biphenylsulfonamide (NXGT191), N′-(2-hydroxy-5-methylbenzylidene)-2-(1-naphthyl)acetohydrazide, 3-{[(4-bromophenyl)amino]sulfonyl}-N-(3-nitrophenyl)benzamide (NXGT1913), or any combinations thereof.

23. The composition of claim 21, wherein the chemical ectopyrase activity inhibitor comprises N-(3-methylphenyl)-4-biphenylsulfonamide (NXGT191).

24. The composition of claim 21, wherein the inhibition of the expression of the APY1, APY2, or both is done by RNA interference (RNAi) using one or more anti-sense or siRNA gene inhibitors.

25. The composition of claim 21, wherein the inhibition of the expression of the APY1, APY2, or both is done by RNA interference (RNAi) selected from SEQ ID NOS: 1-6.

26. A method for increasing a resistance or a tolerance to drought, pathogens or both in a plant comprising the step of administering to a leaf surface of the plant an agricultural composition comprising at least one of a chemical ectopyrase activity inhibitor, a modulator of an ectoapyrase gene transcription, an anti-ectoapyrase antibody or fragments thereof at a concentration sufficient to increase the resistance or the tolerance of the plant to drought, pathogens or both.

27. The method of claim 26, wherein the chemical ectopyrase activity inhibitor comprises NXGT191.

28. The method of claim 26, wherein the inhibition of the expression of the APY1, APY2, or both is done by RNA interference (RNAi) using one or more anti-sense or siRNA gene inhibitors.

29. The method of claim 26, wherein the inhibition of the expression of the APY1, APY2, or both is done by RNA interference (RNAi) selected from SEQ ID NOS: 1-6.

30. A method of conferring increased resistance or tolerance to drought, pathogens or both to a plant comprising the step of:
identifying the plant in need of increased resistance or tolerance to drought, pathogens or both; and
modifying the plant genetically by partially suppressing the transcription of an APY1 gene or an APY2 gene using RNA interference (RNAi), wherein the genetic modification results in a decreased stomatal aperture in the plant resulting in increased resistance or tolerance to drought, pathogens or both.

31. A genetically modified plant with increased resistance or tolerance to drought, pathogens or both made by the method of claim 30.

32. A transgenic plant with increased resistance or tolerance to drought, pathogens or both, wherein the transgenic plant has a decreased transcription of an APY1 gene or an APY2 gene.

33. The transgenic plant of claim 32, wherein the plant has a decreased stomatal aperture.

34. A composition for promoting increased resistance or tolerance to drought pathogens or both comprising:
an extracellular exogenous nucleotides selected from di-nucleotides, tri-nucleotides, or poorly-hydrolyzable nucleotides;
at least one of an ectopyrase activity inhibitor, a modulator of an ectoapyrase gene transcription, an anti-ectoapyrase antibody or fragments, or any combinations thereof; and
a dispersion medium comprising the nucleotides, the inhibitor or both, wherein the dispersion medium comprises an aqueous solvent, an organic solvent, a gas foam, a propellant, or any combinations thereof.

35. The composition of claim 34, wherein the one or more poorly-hydrolyzable nucleotides comprise thio, methylene, amide or methyl-modified ATPγS, ADPβS, ATP, ADP, UTP, UDP, CTP, CDP, TTP, TDP, GTP, GDP, dATP, dADP, dUTP, dUDP, dCTP, dCDP, dTTP, dTDP, dGTP, dGDP, and analogues and combinations thereof.

36. The composition of claim 34, wherein the one or more poorly-hydrolyzable nucleotides comprise ATPγS, ADPβS, or both.

37. A method of increasing resistance or tolerance to drought, pathogens or both in a plant comprising the steps of:
identifying the plant in need of increased resistance to drought, pathogens or both; and
administering to the surface of the plant a composition comprising:
an extracellular exogenous nucleotides selected from di-nucleotides, tri-nucleotides, or poorly-hydrolyzable nucleotides;
at least one of an ectopyrase activity inhibitor, a modulator of an ectoapyrase gene transcription, an anti-ectoapyrase antibody or fragments, or any combinations thereof; and
a dispersion medium comprising the nucleotides, the inhibitor or both, wherein the dispersion medium comprises an aqueous solvent, an organic solvent, a gas foam, a propellant, or any combinations thereof.

38. The method of claim 37, wherein the composition is administered to a surface of the plant, wherein the surface of the plant comprises foliage, leaves, stems, roots, flowers, buds, and stalks.

39. The method of claim 37, wherein the composition is administered to the leaf of the plant.

40. The method of claim 37, wherein the composition is administered by spraying the plant, direct application to the surface of the plant, pouring onto the plant, drenching the root system of the plant, administered as a mixture with soil, water, nutrients, or manure, or any combinations thereof.

41. The method of claim 37, wherein the composition increases the resistance or the tolerance of a plant to the drought, the pathogens, or both by regulating the opening or the closing of one or more stomatal pores.

42. The method of claim 37, wherein the composition modulates extracellular ATP (eATP) mediated cell signaling in the plant.
